(19) United States
(12) Patent Application Publication
(10) Pub. No.: US 2008/0003576 A1
(43) Pub. Date: Jan. 3, 2008

(54) Assay platforms and detection methodology using surface enhanced Raman scattering (SERS) upon specific biochemical interactions

(76) Inventors: Jingwu Zhang, San Jose, CA (US); Handong Li, San Jose, CA (US); Narayan Sundararajan, San Francisco, CA (US); Xing Su, Cupertino, CA (US); Lei Sun, Santa Clara, CA (US)

Correspondence Address: DARBY & DARBY P.C., P.O. BOX 770, Church Street Station, New York, NY, 10008-0770, US

(21) Appl. No.: 11/477,675

(22) Filed: Jun. 30, 2006

(51) Int. Cl. C12Q 1/68; G01N 33/555

(52) U.S. Cl. 435/6; 436/522; 977/902

(57) ABSTRACT

The embodiments of the invention are directed to a SERS cluster comprising a capture particle that is at least partially surrounded by analyte molecules, wherein both the capture particle and the analyte molecules surrounding the capture particle are at least partially surrounded by enhancer particles, wherein a majority of the analyte molecules are either sandwiched between capture and enhancer particles or located between junctions of the enhancer particles. The embodiments of the invention also relate to methods of manufacturing and detecting the SERS cluster. The embodiments of the invention also relate to a SERS active particle comprising a tag molecule comprising a Raman active compound and a probe or a linker having a specific biochemical binding capability and to a method for detecting of a target molecule using a SERS active particle having a tag molecule comprising a Raman active compound and a probe or a linker.

### RELATED APPLICATIONS

This application is related to U.S. Publication 20060033910, published on Feb. 16, 2006, entitled “Composite Organic-Inorganic Nanoparticles (COIN) as SERS tag for analyte detection;” U.S. Publication 20040179195, published on Sep. 16, 2004, entitled “Chemical enhancement in surface enhanced Raman scattering using lithium salts;” U.S. Publication 20050147979, published on Jul. 7, 2005, entitled “Nucleic acid sequencing by Raman monitoring of uptake of nucleotides during molecular replication,” U.S. Publication 20050191665, published on Sep. 1, 2005, entitled “Composite organic-inorganic nanoclusters,” U.S. Publication 20060033910, published on Feb. 16, 2006, entitled “Multiplexed detection of analytes in fluid solution,” and U.S. Ser. No. 11/319,747, filed Dec. 29, 2005, entitled “Modification of metal nanoparticles for improved analyte detection by surface enhanced Raman spectroscopy (SERS),” which are incorporated herein by reference.

### FIELD OF INVENTION

Embodiments of the invention relate to the field of molecular analysis by spectroscopy. The invention relates generally to methods and devices for use in biological, biochemical, and chemical testing, and particularly to methods, instruments, and the use of instruments which utilize new assay platforms and detection methodology using surface enhanced, Raman scattering (SERS) upon specific biochemical interactions.

### BACKGROUND

Raman spectroscopy is a spectroscopic technique used in condensed matter physics, chemistry, biology and medical diagnostics, among others, to study vibrational, rotational, and other low-frequency modes in a system. It relies on inelastic scattering, or Raman scattering of monochromatic light, usually from a laser in the visible, near infrared, or near ultraviolet range. Typically, photons are absorbed or emitted by the laser light, resulting in the energy of the laser photons being shifted up or down. The shift in energy gives information about the phonon modes in the system. Infrared spectroscopy yields similar, but complementary information.

Typically, a sample is illuminated with a laser beam. Light from the illuminated spot is collected with a lens and sent through a monochromator. Wavelengths close to the laser line (due to elastic Rayleigh scattering) are filtered out and those in a certain spectral window away from the laser line are dispersed onto a detector.

Spontaneous Raman scattering is typically very weak, and as a result the main difficulty of Raman spectroscopy is separating the weak inelastically scattered light from the intense Rayleigh scattered laser light. Raman spectrometers typically use holographic diffraction gratings and multiple dispersion stages to achieve a high degree of laser rejection. A photon-counting photomultiplier tube (PMT) or, more commonly, a CCD camera is used to detect the Raman scattered light.

Raman effect occurs when light impinges upon a molecule and interacts with the electron cloud of the bonds of that molecule. The amount of deformation of the electron cloud is the polarizability of the molecule. The amount of the polarizability of the bond could determine the intensity and frequency of the Raman shift. The photon (light quantum), excites one of the electrons into a virtual state. When the photon is released the molecule relaxes back into a vibrational energy state as shown in FIG. 1A. For example, when the molecule relaxes into the zero vibrational energy state (i.e., “ground state”), it generates Rayleigh scattering. The molecule could relax into the first vibration energy states, and this generates Stokes Raman scattering. However, if the molecule was already in an elevated vibrational energy state such as the first vibrational energy state and it relaxes into the zero vibrational energy state, the Raman scattering is then called Anti-Stokes Raman scattering. By Stokes Raman scattering, the wavelength of the emitted light is longer than the wavelength of the excitatory light. By anti-Stokes Raman scattering, the wavelength of the emitted light is shorter that the wavelength of the excitatory light.

Among the many analytical techniques that can be used for chemical structure analysis, surface-enhanced Raman spectroscopy (SERS) is a sensitive method. A Raman spectrum, similar to an infrared spectrum, consists of a wavelength distribution of bands corresponding to molecular vibrations specific to the sample being analyzed. Raman spectroscopy probes vibrational modes of a molecule and the resulting spectrum, similar to an infrared spectrum, is fingerprint-like in nature. To obtain a Raman spectrum, typically a beam from a light source, such as a laser, is focused on the sample generating inelastically scattered radiation which is optically collected and directed into a wavelength-dispersive spectrometer. Although Raman scattering is a relatively low probability event, SERS can be used to enhance signal intensity in the resulting vibrational spectrum.

SERS is a powerful tool that has been demonstrated to detect single molecules. However, the number of molecules that have achieved single molecule level detection is very limited. Moreover, SERS has not been specific to biochemical events. The process of Raman particle aggregation to generate SERS has not been very well controlled. Thus, the sensitive and accurate detection, identification and multiplexed molecular imaging of different chemical/biological composition inside a sample with single molecule sensitivity and high multiplicity have not been done.

Immunoassays like Enzyme-Linked ImmunoSorbent Assay (ELISA) and Polymerase Chain Reaction (PCR) based genetic material detections are currently used. ELISA is a biochemical technique used mainly in immunology to detect the presence of an antibody or an antigen in a sample. It utilizes two antibodies, one of which is specific to the antigen and the other of which is coupled to an enzyme. This second antibody gives the assay its “enzyme-linked” name, and could cause a chromogenic or fluorogenic substrate to produce a signal. Because ELISA can be performed to evaluate either the presence of antigen or the presence of antibody in a sample, it is used both for determining serum antibody concentrations and also for detecting the presence of antigen.

Polymerase chain reaction (PCR) is a molecular biology technique for enzymatically replicating DNA without using a living organism, such as E. coli or yeast. Like amplification using living organisms, the technique allows a small amount of the DNA molecule to be amplified exponentially. However, because it is an in vitro technique, it can be performed without restrictions on the form of DNA and it can be extensively modified to perform a wide array of genetic manipulation. PCR is commonly used in medical and biological research labs for a variety of tasks, such as the detection of hereditary diseases, the identification of genetic fingerprints, the diagnosis of infectious diseases, the cloning of genes, paternity testing, and DNA computing.

Yet, even by ELISA and PCR bases genetic detection techniques, the detection and identification of small numbers (<1000) of molecules from biological and other samples has proven to be an elusive goal, despite widespread potential uses in medical diagnostics, pathology, toxicology, environmental sampling, chemical analysis, forensics and numerous other fields. The embodiments of this invention address these problems in the current state of the art.

### BRIEF DESCRIPTION OF THE DRAWINGS

FIG. 1A shows the vibrational energy states of molecules undergoing Rayleigh scattering, Stokes Raman scattering and anti-Stokes Raman scattering.

FIG. 1B shows SERS cluster formations wherein the position of analyte molecules is not controlled, leading to low average signal intensity as compared with molecules situated at particle junctions.

FIG. 1C shows the SERS spectra of deoxyadenosine-monophosphate (dAMP) using LiCl and NaCl. LiCl generated two times stronger SERS signal of dAMP than NaCl. Spectra were collected for 100 ms with 785 nm excitation. [dAMP]: 9 μM. The final salt concentration was 90 mM. Spectra are plotted offset for comparison. Molecular structure of dAMP is shown in the inset.

FIG. 1D shows SERS spectra of rhodamine 6G. [rhodamine 6G]: 110 nM. Final salt concentration: 90 mM. LiCl generated ten times stronger SERS signal of rhodamine 6G than NaCl. Spectra are plotted offset for comparison. Spectra collected for 100 ms with 785 nm excitation.

FIG. 1E shows the SERS spectra of adenine. [adenine]: 9 nM. Final salt concentration: 90 mM. LiCl generated four times stronger SERS signal of adenine than NaCl. Spectra are plotted offset for comparison. Spectra collected for 100 ms with 785 nm excitation.

FIG. 2 shows three types of functionalized particles: a capture particle, an enhancer particle and a tagged particle.

FIG. 3A shows an embodiment of the invention relating to a new measurement scheme that allows the analyte molecules to be absorbed on the capture particles before more metal particles (enhancer particles) are added to form clusters, thereby providing better control on size and geometry of the SERS cluster.

FIG. 3B shows an embodiment of the invention relating to a method for making capture and enhancer particles to form the SERS cluster based on columbic interaction to bind the capture and enhancer particles.

FIG. 3C shows an embodiment of the invention relating to a method for making capture and enhancer particles to form the SERS cluster based on specific interaction of Biotin-Streptavidin (or Antigen-Antibody).

FIG. 3D shows an embodiment of the invention relating to a method for making capture and enhancer particles to form the SERS cluster based on specific interaction of complimentary DNA (RNA).

FIG. 4A shows an embodiment of the invention for an antigen-antibody specific SERS measurement.

FIG. 4B shows an embodiment of the invention for SERS measurement of a multiplexed assay.

FIG. 4C shows an embodiment of the invention for SERS measurement for cross-detection on one specific target molecule.

FIG. 4D shows an embodiment of the invention for SERS measurement for multiplexed DNA/RNA detection.

### DETAILED DESCRIPTION

As used in the specification and claims, the singular forms “a”, “an” and “the” include plural references unless the context clearly dictates otherwise. For example, the term “an array” may include a plurality of arrays unless the context clearly dictates otherwise.

An “array,” “macroarray” or “microarray” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports. The array could either be a macroarray or a microarray, depending on the size of the sample spots on the array. A macroarray generally contains sample spot sizes of about 300 microns or larger and can be easily imaged by gel and blot scanners. A microarray could generally contain spot sizes of less than 300 microns.

“Solid support,” “support,” and “substrate” refer to a material or group of materials having a rigid or semi-rigid surface or surfaces. In some aspects, at least one surface of the solid support could be substantially flat, although in some aspects it may be desirable to physically separate synthesis regions for different molecules with, for example, wells, raised regions, pins, etched trenches, or the like. In certain aspects, the solid support(s) could take the form of beads, resins, gels, microspheres, or other geometric configurations.

A “nanomaterial” as used herein refers to a structure, a device or a system having a dimension at the atomic, molecular or macromolecular levels, in the length scale of approximately 1-100 nanometer range. Preferably, a nanomaterial has properties and functions because of the size and can be manipulated and controlled on the atomic level.

The term “target” or “target molecule” refers to a molecule of interest that is to be analyzed, e.g., a nucleotide, an oligonucleotide, or a protein. The target or target molecule could be a small molecule, biomolecule, or nanomaterial such as but not necessarily limited to a small molecule that is biologically active, nucleic acids and their sequences, peptides and polypeptides, as well as nanostructure materials chemically modified with biomolecules or small molecules capable of binding to molecular probes such as chemically modified carbon nanotubes, carbon nanotube bundles, nanowires, nanoclusters or nanoparticles. The target molecule may be fluorescently labeled DNA or RNA.

The term “probe” or “probe molecule” refers to a molecule that binds to a target molecule for the analysis of the target. The probe or probe molecule is generally, but not necessarily, has a known molecular structure or sequence. The probe or probe molecule is generally, but not necessarily, attached to the substrate of the array. The probe or probe molecule is typically a nucleotide, an oligonucleotide, or a protein, including, for example, cDNA or pre-synthesized polynucleotide deposited on the array. Probes molecules are biomolecules capable of undergoing binding or molecular recognition events with target molecules. (In some references, the terms “target” and “probe” are defined opposite to the definitions provided here.) The polynucleotide probes require the sequence information of genes, and thereby can exploit the genome sequences of an organism. In cDNA arrays, there could be cross-hybridization due to sequence homologies among members of a gene family. Polynucleotide arrays can be specifically designed to differentiate between highly homologous members of a gene family as well as spliced forms of the same gene (exon-specific). Polynucleotide arrays of the embodiment of this invention could also be designed to allow detection of mutations and single nucleotide polymorphism. A probe or probe molecule can be a capture molecule.

The term “molecule” generally refers to a macromolecule or polymer as described herein. However, arrays comprising single molecules, as opposed to macromolecules or polymers, are also within the scope of the embodiments of the invention.

A “macromolecule” or “polymer” comprises two or more monomers covalently joined. The monomers may be joined one at a time or in strings of multiple monomers, ordinarily known as “oligomers.” Thus, for example, one monomer and a string of five monomers may be joined to form a macromolecule or polymer of six monomers. Similarly, a string of fifty monomers may be joined with a string of hundred monomers to form a macromolecule or polymer of one hundred and fifty monomers. The term polymer as used herein includes, for example, both linear and cyclic polymers of nucleic acids, polynucleotides, polynucleotides, polysaccharides, oligosaccharides, proteins, polypeptides, peptides, phospholipids and peptide nucleic acids (PNAs). The peptides include those peptides having either α-, β-, or ω-amino acids. In addition, polymers include heteropolymers in which a known drug is covalently bound to any of the above, polyurethanes, polyesters, polycarbonates, polyureas, polyamides, polyethyleneimines, polyarylene sulfides, polysiloxanes, polyimides, polyacetates, or other polymers which could be apparent upon review of this disclosure.

The term “nucleotide” includes deoxynucleotides and analogs thereof. These analogs are those molecules having some structural features in common with a naturally occurring nucleotide such that when incorporated into a polynucleotide sequence, they allow hybridization with a complementary polynucleotide in solution. Typically, these analogs are derived from naturally occurring nucleotides by replacing and/or modifying the base, the ribose or the phosphodiester moiety. The changes can be tailor-made to stabilize or destabilize hybrid formation, or to enhance the specificity of hybridization with a complementary polynucleotide sequence as desired, or to enhance stability of the polynucleotide.

The term “polynucleotide” or “nucleic acid” as used herein refers to a polymeric form of nucleotides of any length, either ribonucleotides or deoxyribonucleotides, that comprise purine and pyrimidine bases, or other natural, chemically or biochemically modified, non-natural, or derivatized nucleotide bases. Polynucleotides of the embodiments of the invention include sequences of deoxyribopolynucleotide (DNA), ribopolynucleotide (RNA), or DNA copies of ribopolynucleotide (cDNA) which may be isolated from natural sources, recombinantly produced, or artificially synthesized. A further example of a polynucleotide of the embodiments of the invention may be polyamide polynucleotide (PNA). The polynucleotides and nucleic acids may exist as single-stranded or double-stranded. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. The polymers made of nucleotides such as nucleic acids, polynucleotides and polynucleotides may also be referred to herein as “nucleotide polymers.

An “oligonucleotide” is a polynucleotide having 2 to 20 nucleotides. Analogs also include protected and/or modified monomers as are conventionally used in polynucleotide synthesis. As one of skill in the art is well aware, polynucleotide synthesis uses a variety of base-protected nucleoside derivatives in which one or more of the nitrogens of the purine and pyrimidine moiety are protected by groups such as dimethoxytrityl, benzyl, tert-butyl, isobutyl and the like.

When the macromolecule of interest is a peptide, the amino acids can be any amino acids, including α, β, or ω-amino acids. When the amino acids are α-amino acids, either the L-optical isomer or the D-optical isomer may be used. Additionally, unnatural amino acids, for example, β-alanine, phenylglycine and homoarginine are also contemplated by the embodiments of the invention. These amino acids are well-known in the art.

A “peptide” is a polymer in which the monomers are amino acids and which are joined together through amide bonds and alternatively referred to as a polypeptide. In the context of this specification it should be appreciated that the amino acids may be the L-optical isomer or the D-optical isomer. Peptides are two or more amino acid monomers long, and often more than 20 amino acid monomers long.

A “protein” is a long polymer of amino acids linked via peptide bonds and which may be composed of two or more polypeptide chains. More specifically, the term “protein” refers to a molecule composed of one or more chains of amino acids in a specific order; for example, the order as determined by the base sequence of nucleotides in the gene coding for the protein. Proteins are essential for the structure, function, and regulation of the body's cells, tissues, and organs, and each protein has unique functions. Examples are hormones, enzymes, and antibodies.

The term “sequence” refers to the particular ordering of monomers within a macromolecule and it may be referred to herein as the sequence of the macromolecule.

The term “hybridization” refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.” For example, hybridization refers to the formation of hybrids between a probe polynucleotide (e.g., a polynucleotide of the invention which may include substitutions, deletion, and/or additions) and a specific target polynucleotide (e.g., an analyte polynucleotide) wherein the probe preferentially hybridizes to the specific target polynucleotide and substantially does not hybridize to polynucleotides consisting of sequences which are not substantially complementary to the target polynucleotide. However, it could be recognized by those of skill that the minimum length of a polynucleotide desired for specific hybridization to a target polynucleotide could depend on several factors: G/C content, positioning of mismatched bases (if any), degree of uniqueness of the sequence as compared to the population of target polynucleotides, and chemical nature of the polynucleotide (e.g., methylphosphonate backbone, phosphorothiolate, etc.), among others.

Methods for conducting polynucleotide hybridization assays have been well developed in the art. Hybridization assay procedures and conditions could vary depending on the application and are selected in accordance with the general binding methods known in the art.

It is appreciated that the ability of two single stranded polynucleotides to hybridize could depend upon factors such as their degree of complementarity as well as the stringency of the hybridization reaction conditions.

As used herein, “stringency” refers to the conditions of a hybridization reaction that influence the degree to which polynucleotides hybridize. Stringent conditions can be selected that allow polynucleotide duplexes to be distinguished based on their degree of mismatch. High stringency is correlated with a lower probability for the formation of a duplex containing mismatched bases. Thus, the higher the stringency, the greater the probability that two single-stranded polynucleotides, capable of forming a mismatched duplex, could remain single-stranded. Conversely, at lower stringency, the probability of formation of a mismatched duplex is increased.

The appropriate stringency that could allow selection of a perfectly-matched duplex, compared to a duplex containing one or more mismatches (or that could allow selection of a particular mismatched duplex compared to a duplex with a higher degree of mismatch) is generally determined empirically. Means for adjusting the stringency of a hybridization reaction are well-known to those of skill in the art.

A “ligand” is a molecule that is recognized by a particular receptor. Examples of ligands that can be investigated by this invention include, but are not restricted to, agonists and antagonists for cell membrane receptors, toxins and venoms, viral epitopes, hormones, hormone receptors, peptides, enzymes, enzyme substrates, cofactors, drugs (e.g. opiates, steroids, etc.), lectins, sugars, polynucleotides, nucleic acids, oligosaccharides, proteins, and monoclonal antibodies.

A “receptor” is molecule that has an affinity for a given ligand. Receptors may-be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term “receptors” is used herein, no difference in meaning is intended. A “ligand receptor pair” is formed when two macromolecules have combined through molecular recognition to form a complex. Other examples of receptors which can be investigated by this invention include but are not restricted to:

a) Microorganism receptors: Determination of ligands which bind to receptors, such as specific transport proteins or enzymes essential to survival of microorganisms, is useful in developing a new class of antibiotics. Of particular value could be antibiotics against opportunistic fungi, protozoa, and those bacteria resistant to the antibiotics in current use.

b) Enzymes: For instance, one type of receptor is the binding site of enzymes such as the enzymes responsible for cleaving neurotransmitters; determination of ligands which bind to certain receptors to modulate the action of the enzymes which cleave the different neurotransmitters is useful in the development of drugs which can be used in the treatment of disorders of neurotransmission.

c) Antibodies: For instance, the invention may be useful in investigating the ligand-binding site on the antibody molecule which combines with the epitope of an antigen of interest; determining a sequence that mimics an antigenic epitope may lead to the-development of vaccines of which the immunogen is based on one or more of such sequences or lead to the development of related diagnostic agents or compounds useful in therapeutic treatments such as for auto-immune diseases (e.g., by blocking the binding of the “anti-self” antibodies).

d) Nucleic Acids: Sequences of nucleic acids may be synthesized to establish DNA or RNA binding sequences.

e) Catalytic Polypeptides: Polymers, preferably polypeptides, which are capable of promoting a chemical reaction involving the conversion of one or more reactants to one or more products. Such polypeptides generally include a binding site specific for at least one reactant or reaction intermediate and an active functionality proximate to the binding site, which functionality is capable of chemically modifying the bound reactant.

f) Hormone receptors: Examples of hormones receptors include, e.g., the receptors for insulin and growth hormone. Determination of the ligands which bind with high affinity to a receptor is useful in the development of, for example, an oral replacement of the daily injections which diabetics take to relieve the symptoms of diabetes. Other examples are the vasoconstrictive hormone receptors; determination of those ligands which bind to a receptor may lead to the development of drugs to control blood pressure.

g) Opiate receptors: Determination of ligands which bind to the opiate receptors in the brain is useful in the development of less-addictive replacements for morphine and related drugs.

The term “specific binding” or “specific interaction” is the specific recognition of one of two different molecules for the other compared to substantially less recognition of other molecules. Generally, the molecules have areas on their surfaces or in cavities giving rise to specific recognition between the two molecules. Exemplary of specific binding are antibody-antigen interactions, enzyme—substrate interactions, polynucleotide hybridization interactions, and so forth.

The term “bi-functional linker group” refers to an organic chemical compound that has at least two chemical groups or moieties, such are, carboxyl group, amine group, thiol group, aldehyde group, epoxy group, that can be covalently modified specifically; the distance between these groups is equivalent to or greater than 5-carbon bonds.

The phrase “SERS active material,” “SERS active particle,” or “SERS cluster” refers to a material, a particle or a cluster of particles that produces a surface-enhanced Raman scattering effect. The SERS active material or particle generates surface enhanced Raman signal specific to the analyte molecules when the analyte-particle complexes are excited with a light source as compared to the Raman signal from the analyte alone in the absence of the SERS active material or SERS active particle. The enhanced Raman scattering effect provides a greatly enhanced Raman signal from Raman-active analyte molecules that have been adsorbed onto certain specially-prepared SERS active surfaces. The SERS active surface could be planar or curved. Typically, the SERS active surfaces are metal surfaces. Increases in the intensity of Raman signal could be in the order of 10⁴-10¹⁴ for some systems. SERS active material or particle includes a variety of metals including coinage (Au, Ag, Cu), alkalis (Li, Na, K), Al, Pd and Pt. In the case of SERS active particle, the particle size of SERS active particles could range from 1 to 5000 nanometers, preferably in the range of 5 to 250 nanometers, more preferably in the range of 10 to 150 nanometers, and most preferably 40 to 80 nanometers.

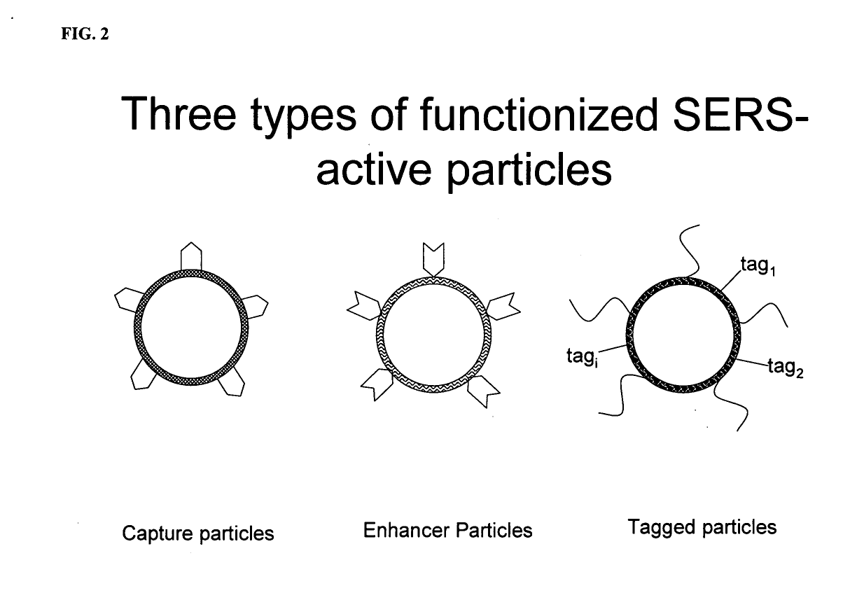

The term “capture particle” refers to a particle that can capture an analyte. The capture particle could be a coinage metal nanoparticle with surface modification to allow strong physical and/or chemical adsorption of analyte molecules and to allow adhesion of “enhancer particles” by electrostatic attraction, through specific interaction using a linker such as antibody-antigen, DNA hybridization, etc. or through the analyte molecule. An embodiment of a capture particle is shown in FIG. 2 wherein a metal particle has surface modification (shown as a hatched ring) and further has linkers that can combine with linkers on an enhancer particle.

The term “enhancer particle” refers to a SERS active particle with suitable surface modification, a linker or an analyte which combines with a capture particle to form an aggregate. In case the capture particle is positively charged, then a negatively charged SERS active particle can be used as an enhancer particle without a linker, and vise versa. In case the capture particle has an antigen or an antibody, then a SERS active particle having a complimentary linker, namely, an antibody or an antigen, could be used as an enhancer particle. An embodiment of an enhancer particle is shown in FIG. 2 wherein a metal particle has surface modification (shown as a hatched ring) and further has linkers that can combine with linkers on a capture particle.

The term “tagged particle” refers a SERS active particle having one or more different Raman active labels attached to the SERS active particle by direct attachment or through a surface modification. A tagged particle has a linker that can link to another tagged particle via an analyte. An embodiment of a tagged particle is shown in FIG. 2 wherein a metal particle has surface modification (shown as a hatched ring) and further has Raman active labels and linkers that can link to another tagged particle via an analyte.

As used herein, the term “colloid” refers to nanometer size metal particles suspending in a liquid, usually an aqueous solution. In the methods of the invention, the colloidal particles are prepared by mixing metal cations and reducing agent in aqueous solution prior to heating. Typical metals contemplated for use in the practice of the invention include, for example, silver, gold, platinum, copper, and the like. A variety of reducing agents are contemplated for use in the practice of the invention, such as, for example, citrate, borohydride, ascorbic acid and the like. Sodium citrate is used in certain embodiments of the invention. Typically, the metal cations and reducing agent are each present in aqueous solution at a concentration of at least about 0.5 mM. After mixing the metal cations and reducing agent, the solution is heated for about 30 minutes. In some embodiments, the solution is heated for about 60 minutes. Typically, the solution is heated to about 95° C. In other embodiments, the solution is heated to about 100° C. Heating of the solution is accomplished in a variety of ways well known to those skilled in the art. In some embodiments, the heating is accomplished using a microwave oven, a convection oven, or a combination thereof. The methods for producing metallic colloids described herein are in contrast to prior methods wherein a boiling silver nitrate solution is titrated with a sodium citrate solution. This titration method can produce one batch of silver particles with adequate Raman enhancement to dAMP in about 10 attempts, and the other batches have low or no Raman activity at all. However, by employing the methods of the invention, an average SERS signal enhancement of 150% is observed relative to colloids prepared from the titration method.

The metallic colloids could be modified by attaching an organic molecule to the surface of the colloids. Organic molecules contemplated would typically be less than about 500 Dalton in molecular weight, and are bifunctional organic molecules. As used herein, a “bifunctional organic molecule” means that the organic molecule has a moiety that has an affinity for the metallic surface, and a moiety that has an affinity for a biomolecule. Such surface modified metallic colloids exhibit an increased ability to bind biomolecules, thereby resulting in an enhanced and reproducible SERS signal. The colloids can be used either individually, or as aggregates for binding certain biomolecules.

Organic molecules contemplated for use include molecules having any moiety that exhibits an affinity for the metals contemplated for use in the methods of the invention (i.e., silver, gold, platinum, copper, aluminum, and the like), and any moiety that exhibit affinities for biomolecules. For example, with regard to silver or gold affinity, in some embodiments, the organic molecule has a sulfur containing moiety, such as for example, thiol, disulfide, and the like. With regard to affinity for a biomolecule such as a polynucleotide, for example, the organic molecule has a carboxylic acid moiety. In certain embodiments, the organic molecule is thiomalic acid, L-cysteine diethyl ester, S-carboxymethyl-L-cysteine, cystamine, meso-2,3-dimercaptosuccinic acid, and the like. It is understood, however, that any organic molecule that meets the definition of a “bifunctional organic molecule”, as described herein, is contemplated for use in the practice of the invention. It is also understood that the organic molecule may be attached to the metallic surface and the biomolecule either covalently, or non-covalently. Indeed, the term “affinity” is intended to encompass the entire spectrum of chemical bonding interactions.

This surface modification of metallic colloids provides certain advantages in SERS detection analyses. For example, the surfaces of the metallic colloids could be tailored to bind to a specific biomolecule or the surfaces can be tailored to differentiate among groups of proteins based on the side chains of the individual amino acid residues found in the protein.

The term “COIN” refers to a composite-organic-inorganic nanoparticle(s). The COIN could be surface-enhanced Raman scattering (SERS, also referred to as surface-enhanced Raman spectroscopy)-active nanoclusters incorporated into a gel matrix and used in certain other analyte separation techniques described herein.

COINs are composite organic-inorganic nanoclusters. The clusters include several fused or aggregated metal particles with a Raman-active organic compound adsorbed on the metal particles and/or in the junctions of the metal particles. Organic Raman labels can be incorporated into the coalescing metal particles to form stable clusters and produce intrinsically enhanced Raman scattering signals. The interaction between the organic Raman label molecules and the metal colloids has mutual benefits. Besides serving as signal sources, the organic molecules promote and stabilize metal particle association that is in favor of SERS. On the other hand, the metal particles provide spaces to hold and stabilize Raman label molecules, especially in the cluster junctions.

These SERS-active probe constructs comprise a core and a surface, wherein the core comprises a metallic colloid comprising a first metal and a Raman-active organic compound. The COINs can further comprise a second metal different from the first metal, wherein the second metal forms a layer overlying the surface of the nanoparticle. The COINs can further comprise an organic layer overlying the metal layer, which organic layer comprises the probe. Suitable probes for attachment to the surface of the SERS-active nanoclusters include, without limitation, antibodies, antigens, polynucleotides, oligonucleotides, receptors, ligands, and the like.

The metal required for achieving a suitable SERS signal is inherent in the COIN, and a wide variety of Raman-active organic compounds can be incorporated into the particle. Indeed, a large number of unique Raman signatures can be created by employing nanoclusters containing Raman-active organic compounds of different structures, mixtures, and ratios. Thus, the methods described herein employing COINs are useful for the simultaneous detection of many multiple components such as analytes in a sample, resulting in rapid qualitative analysis of the contents of “profile” of a body fluid. In addition, since many COINs can be incorporated into a single nanoparticle, the SERS signal from a single COIN particle is strong relative to SERS signals obtained from Raman-active materials that do not contain the nanoclusters described herein as COINs. This situation results in increased sensitivity compared to Raman-techniques that do not utilize COINs.

COINs could be prepared using standard metal colloid chemistry. The preparation of COINs also takes advantage of the ability of metals to adsorb organic compounds. Indeed, since Raman-active organic compounds are adsorbed onto the metal during formation of the metallic colloids, many Raman-active organic compounds can be incorporated into the COIN without requiring special attachment chemistry.

In general, the COINs could be prepared as follows. An aqueous solution is prepared containing suitable metal cations, a reducing agent, and at least one suitable Raman-active organic compound. The components of the solution are then subject to conditions that reduce the metallic cations to form neutral, colloidal metal particles. Since the formation of the metallic colloids occurs in the presence of a suitable Raman-active organic compound, the Raman-active organic compound is readily adsorbed onto the metal during colloid formation. COINs of different sizes can be enriched by centrifugation.

Typically, organic compounds are attached to a layer of a second metal in COINs by covalently attaching organic compounds to the surface of the metal layer Covalent attachment of an organic layer to the metallic layer can be achieved in a variety ways well known to those skilled in the art, such as, for example, through thiol-metal bonds. In alternative embodiments, the organic molecules attached to the metal layer can be crosslinked to form a molecular network.

The COIN(s) can include cores containing magnetic materials, such as, for example, iron oxides, and the like such that the COIN is a magnetic COIN. Magnetic COINs can be handled without centrifugation using commonly available magnetic particle handling systems. Indeed, magnetism can be used as a mechanism for separating biological targets attached to magnetic COIN particles tagged with particular biological probes.

The term “reporter” means a detectable moiety. The reporter can be detected, for example, by Raman spectroscopy. Generally, the reporter and any molecule linked to the reporter can be detected without a second binding reaction. The reporter can be COIN (composite-organic-inorganic nanoparticle), magnetic-COIN, quantum dots, and other Raman or fluorescent tags, for example.

As used herein, “Raman-active organic compound” refers to an organic molecule that produces a unique SERS signature in response to excitation by a laser. A variety of Raman-active organic compounds are contemplated for use as components in COINs. In certain embodiments, Raman-active organic compounds are polycyclic aromatic or heteroaromatic compounds. Typically the Raman-active organic compound has a molecular weight less than about 300 Daltons.

Additional, non-limiting examples of Raman-active organic compounds useful in COINs include TRIT (tetramethyl rhodamine isothiol), NBD (7-nitrobenz-2-oxa-1,3-diazole), Texas Red dye, phthalic acid, terephthalic acid, isophthalic acid, cresyl fast violet, cresyl blue violet, brilliant cresyl blue, para-aminobenzoic acid, erythrosine, biotin, digoxigenin, 5-carboxy-4′,5′-dichloro-2′,7′-dimethoxy fluorescein, 5-carboxy-2′,4′,5′,7′-tetrachlorofluorescein, 5-carboxyfluorescein, 5-carboxy rhodamine, 6-carboxyrhodamine, 6-carboxytetramethyl amino phthalocyanines, azomethines, cyanines, xanthines, succinylfluoresceins, aminoacridine, and the like.

In certain embodiments, the Raman-active compound is adenine, adenine, 4-amino-pyrazolo(3,4-d)pyrimidine, 2-fluoroadenine, N6-benzolyadenine, kinetin, dimethyl-allyl-amino-adenine, zeatin, bromo-adenine, 8-aza-adenine, 8-azaguanine, 6-mercaptopurine, 4-amino-6-mercaptopyrazolo(3,4-d)pyrimidine, 8-mercaptoadenine, or 9-amino-acridine4-amino-pyrazolo(3,4-d)pyrimidine, or 2-fluoroadenine. In one embodiment, the Raman-active compound is adenine.

When “fluorescent compounds” are incorporated into COINs, the fluorescent compounds can include, but are not limited to, dyes, intrinsically fluorescent proteins, lanthanide phosphors, and the like. Dyes useful for incorporation into COINs include, for example, rhodamine and derivatives, such as Texas Red, ROX (6-carboxy-X-rhodamine), rhodamine-NHS, and TAMRA (5/6-carboxyletramethyl rhodamine NHS); fluorescein and derivatives, such as 5-bromomethyl fluorescein and FAM (5′-carboxyfluorescein NHS), Lucifer Yellow, IAEDANS, 7-Me₂, N-coumarin-4-acetate, 7-OH-4-CH₃-coumarin-3-acetate, 7-NH₂-4CH₃-coumarin-3-acetate (AMCA), monobromobimane, pyrene trisulfonates, such as Cascade Blue, and monobromotrimethyl-ammoniobimane.

Multiplex testing of a complex sample could generally be based on a coding system that possesses identifiers for a large number of reactants in the sample. The primary variable that determines the achievable numbers of identifiers in currently known coding systems is, however, the physical dimension. Tagging techniques, based on surface-enhanced Raman scattering (SERS) of fluorescent dyes, could be used in the embodiments of this invention for developing chemical structure-based coding systems.

COINs may be used to detect the presence of a particular target analyte, for example, a nucleic acid, oligonucleotide, protein, enzyme, antibody or antigen. The nanoclusters may also be used to screen bioactive agents, i.e. drug candidates, for binding to a particular target or to detect agents like pollutants. Any analyte for which a probe moiety, such as a peptide, protein, oligonucleotide or aptamer, may be designed can be used in combination with the disclosed nanoclusters.

Also, SERS-active COINs that have an antibody as binding partner could be used to detect interaction of the Raman-active antibody labeled constructs with antigens either in solution or on a solid support. It could be understood that such immunoassays can be performed using known methods such as are used, for example, in ELISA assays, Western blotting, or protein arrays, utilizing a SERS-active COIN having an antibody as the probe and acting as either a primary or a secondary antibody, in place of a primary or secondary antibody labeled with an enzyme or a radioactive compound. In another example, a SERS-active COIN is attached to an enzyme probe for use in detecting interaction of the enzyme with a substrate.

Another group of exemplary methods could use the SERS-active COINs to detect a target nucleic acid. Such a method is useful, for example, for detection of infectious agents within a clinical sample, detection of an amplification product derived from genomic DNA or RNA or message RNA, or detection of a gene (cDNA) insert within a clone. For certain methods aimed at detection of a target polynucleotide, an oligonucleotide probe is synthesized using methods known in the art. The oligonucleotide is then used to functionalize a SERS-active COIN. Detection of the specific Raman label in the SERS-active COIN identifies the nucleotide sequence of the oligonucleotide probe, which in turn provides information regarding the nucleotide sequence of the target polynucleotide.

The term “complementary” refers to the topological compatibility or matching together of interacting surfaces of a ligand molecule and its receptor. Thus, the receptor and its ligand can be described as complementary, and furthermore, the contact surface characteristics are complementary to each other.

The terms “spectrum” or “spectra” refer to the intensities of electromagnetic radiation as a function of wavelength or other equivalent units, such as wavenumber, frequency, and energy level.

The term “spectrometer” refers to an instrument equipped with scales for measuring wavelengths or indexes of refraction.

The term “dispersive spectrometer” refers to a spectrometer that generates spectra by optically dispersing the incoming radiation into its frequency or spectral components. Dispersive spectrometers can be further classified into two types: monochromators and spectrographs. A monochromator uses a single detector, narrow slit(s) (usually two, one at the entrance and another at the exit port), and a rotating dispersive element allowing the user to observe a selected range of wavelength. A spectrograph, on the other hand, uses an array of detector elements and a stationary dispersive element. In this case, the slit shown in the figure is removed, and spectral elements over a wide range of wavelengths are obtained at the same time, therefore providing faster measurements with a more expensive detection system.

The term “analyte” means any atom, chemical, molecule, compound, composition or aggregate of interest for detection and/or identification. Examples of analytes include, but are not limited to, an amino acid, peptide, polypeptide, protein, glycoprotein, lipoprotein, nucleoside, nucleotide, oligonucleotide, nucleic acid, sugar, carbohydrate, oligosaccharide, polysaccharide, fatty acid, lipid, hormone, metabolite, cytokine, chemokine, receptor, neurotransmitter, antigen, allergen, antibody, substrate, metabolite, cofactor, inhibitor, drug, pharmaceutical, nutrient, prion, toxin, poison, explosive, pesticide, chemical warfare agent, biohazardous agent, radioisotope, vitamin, heterocyclic aromatic compound, carcinogen, mutagen, narcotic, amphetamine, barbiturate, hallucinogen, waste product and/or contaminant. In certain embodiments of the invention, one or more analytes may be labeled with one or more Raman labels, as disclosed below. The sample such as an analyte in the embodiments of this invention could be in the form of solid, liquid or gas. The sample could be analyzed by the embodiments of the method and device of this invention when the sample is at room temperature and at lower than or higher than the room temperature.

The term “label” or “tag” is used to refer to any molecule, compound or composition that can be used to identify a sample such as an analyte to which the label is attached. In various embodiments of the invention, such attachment may be either covalent or non-covalent. In non-limiting examples, labels may be fluorescent, phosphorescent, luminescent, electroluminescent, chemiluminescent or any bulky group or may exhibit Raman or other spectroscopic characteristics.

A “Raman label” or “Raman tag” may be any organic or inorganic molecule, atom, complex or structure capable of producing a detectable Raman signal, including but not limited to synthetic molecules, dyes, naturally occurring pigments such as phycoerythrin, organic nanostructures such as C₆₀, buckyballs and carbon nanotubes, metal nanostructures such as gold or silver nanoparticles or nanoprisms and nano-scale semiconductors such as quantum dots. Numerous examples of Raman labels are disclosed below. A person of ordinary skill in the art could realize that such examples are not limiting, and that “Raman label” encompasses any organic or inorganic molecule, compound or structure known in the art that can be detected by Raman spectroscopy.

The term “fluid” used herein means an aggregate of matter that has the tendency to assume the shape of its container, for example a liquid or gas. Analytes in fluid form can include fluid suspensions and solutions of solid particle analytes.

The term “majority” means more than 50 percent.

When light passes through a medium of interest, a certain amount becomes diverted from its original direction. This phenomenon is known as scattering. Some of the scattered light differs in frequency from the original excitatory light, due to a) the absorption of light by the medium, b) excitation of electrons in the medium to a higher energy state, and c) subsequent emission of the light from the medium at a different wavelength. When the frequency difference matches the energy level of the molecular vibrations of the medium of interest, this process is known as Raman scattering. The wavelengths of the Raman emission spectrum are characteristic of the chemical composition and structure of the molecules absorbing the light in a sample, while the intensity of light scattering is dependent on the concentration of molecules in the sample as well as the structure of the molecule.

Typically, the probability of Raman interaction occurring between an excitatory light beam and an individual molecule in a sample is very low, resulting in a low sensitivity. The term “optical cross section” indicates the probability of an optical event occurring in a particular molecule or a particle. When photons impinge on a molecule, some of the photons that geometrically impinge on the molecule interact with the electron cloud of the molecule. The term “geometric cross-section” is the volume per molecule in which the photons interact with the electron cloud of the molecule. The term “cross section” is the product of the geometric cross-section and the optical cross section. Optical detection and spectroscopy techniques of a single molecule require cross sections greater than 10⁻²¹ cm²/molecule, more preferably cross-sections greater than 10⁻¹⁶ cm²/molecule. On the other hand, typical spontaneous Raman scattering techniques have cross sections of about 10⁻³⁰ cm²/molecule, and thus are not suitable for single molecule detection.

In SERS, molecules located near metal are excited by the surface plasmon generated by interaction between the excitation light and the metallic surface. Specifically, it has been observed that molecules near roughened silver surfaces show enhanced Raman scattering of as much as six to seven orders of magnitude. The SERS effect is related to the phenomenon of plasmon resonance, wherein a metal surface exhibits a pronounced optical resonance in response to incident electromagnetic radiation, due to the collective coupling of conduction electrons in the metal. In essence, metal surface can function as miniature “dish-antenna” to enhance the localized effects of electromagnetic radiation. Molecules located in the vicinity of such surfaces exhibit a much greater sensitivity for Raman spectroscopic analysis. In ideal condition, the surface plasmon has several orders of magnitude higher intensity of electromagnetic field compared to the intensity of electromagnetic field of excitation light, and hence the Raman scattering by the molecules are several orders stronger than what the excitation light could have generated without the surface enhancements.

SERS techniques can give strong intensity enhancements by a factor of up to 10¹⁴ to 10¹⁶ or 10¹⁸, preferably for certain molecules (for example, dye molecules, adenine, hemoglobin, and tyrosine), which is near the range of single molecule detection. Generally, SERS is observed for molecules found close to silver or gold nanoparticles (although other metals may be used, but with a reduction in enhancement). The mechanism by which the enhancement of the Raman signal is provided is from a local electromagnetic field enhancement provided by an optically active nanoparticle. Current understanding suggests that the enhanced optical activity results from the excitation of local surface plasmon modes that are excited by focusing laser light onto the nanoparticle. SERS gives all the information usually found in Raman spectra; it is a sensitive vibrational spectroscopy that gives structural information on the molecule and its local interactions.

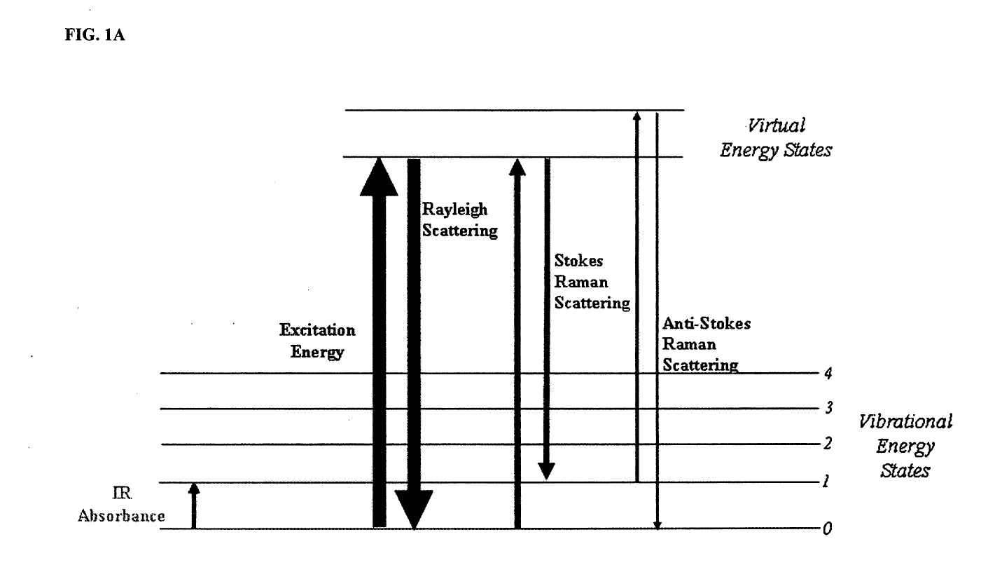

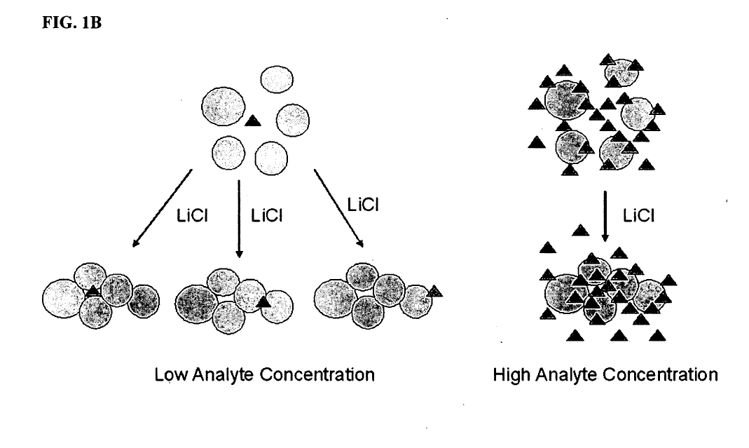

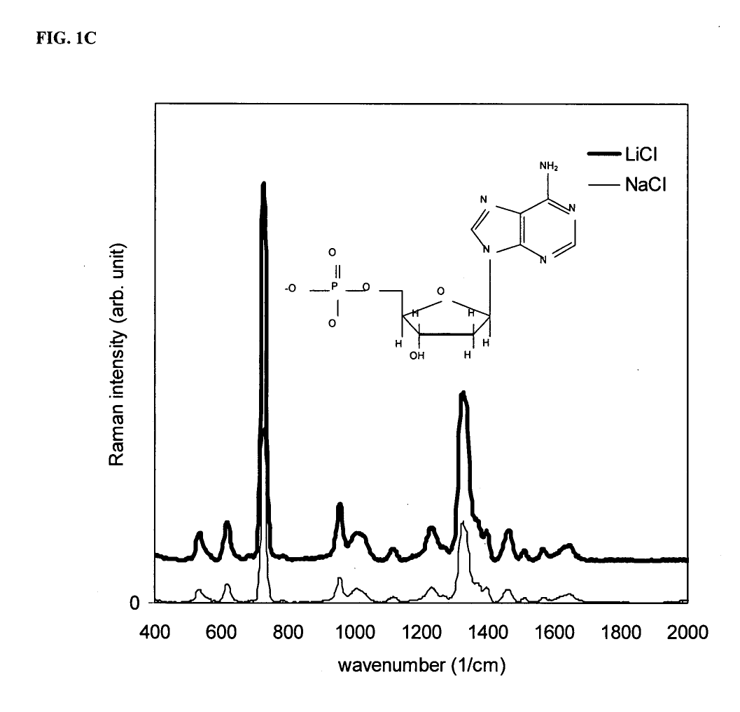

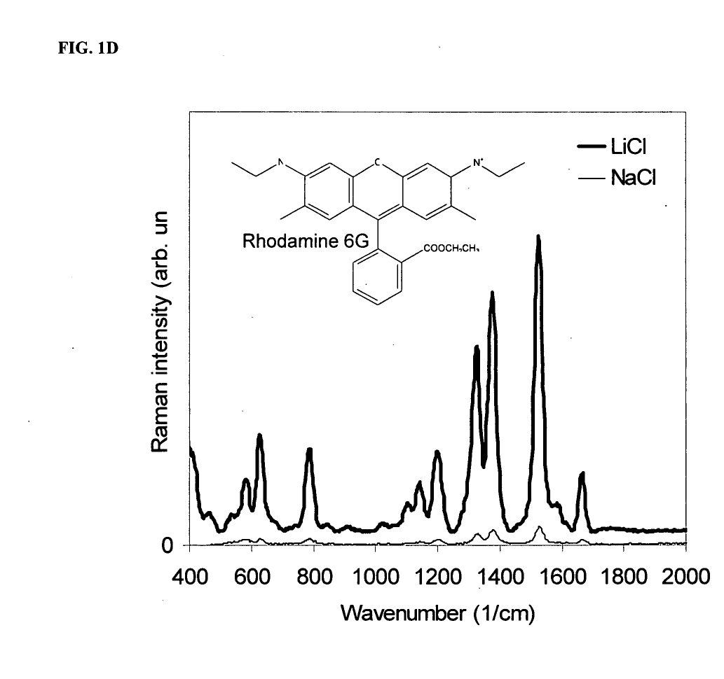

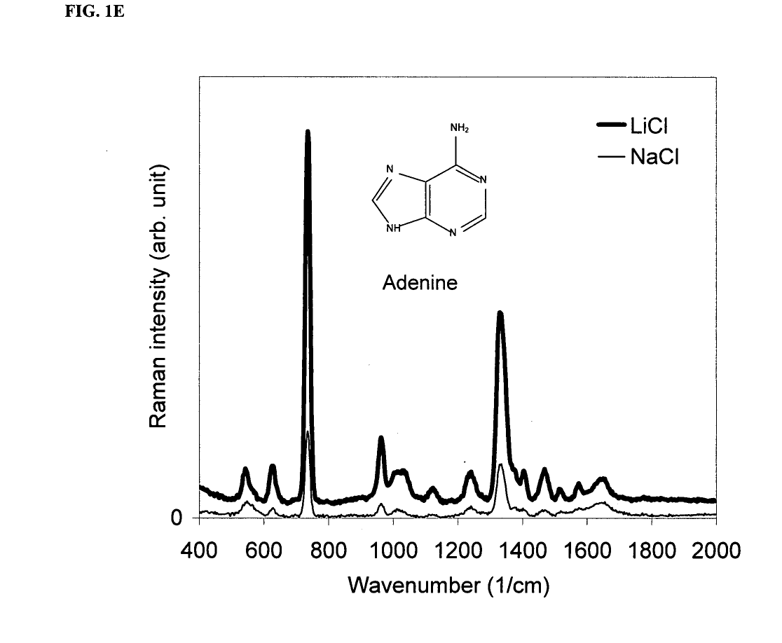

The signal enhancement is much stronger for analyte molecules, which are shown as triangles in FIG. 1B, that are situated at the junctions of nanoparticles, which are shown as circles in FIG. 1B, as compared with the analyte molecules absorbed on the free surface of the metal particles. For example, in the low analyte concentration case of FIG. 1B, the analyte molecule within the cluster of nanoparticles shown in the left side of the figure entitled “Low Analyte Concentration” would have a greater signal enhancement as compared to the analyte molecule absorbed on the free surface of the nanoparticles of the clusters shown in the middle and right sides of the same figure. In the SERS measurement scheme of FIG. 1B, particularly for the case of high analyte concentration, most of the analyte molecules are situated at positions where the electromagnetic effect is relatively low, which could lead to low average signal intensity. By the embodiments of the invention, the applicants have developed a new SERS measurement scheme to enrich analyte molecules between metal particle junctions, thereby to improve the SERS signal intensity and the detection limit for any types of analytes.

By the embodiments of this invention, the SERS techniques could be used such that cross sections of up to about 10⁻¹ to 10⁻¹⁶ cm²/molecule could be consistently observed for a wide range of molecules. Enhancements in this range could be in the range of single molecule detection. For example, the SERS techniques could be in combination with coherent anti-Raman spectroscopy (CARS), such as surface enhanced coherent anti-Stokes Raman spectroscopy (hereinafter SECARS), to allow for single molecule detection. CARS techniques alone could give intensity enhancement by a factor of about 10⁵ which yields cross sections in the range of about 10⁻²⁵ cm²/molecule, still too small for optical detection and spectroscopy of single molecules. However, the new assay platforms users SERS by the embodiments of this invention could provide enhancements by a factor of 10⁹ to 10¹⁸ or greater, preferably by fine tuning the assay for each type of molecule.

The embodiments of the invention relate to new platforms for detecting biomolecules based on SERS of tag molecules induced by specific interactions between target and probe molecules. For example, the platforms could specifically detect target molecules such as proteins, viruses, and nucleic acids using SERS.

By the embodiments of the invention, specific biochemical interactions could be designed to cause event-specific Raman particle aggregation to generate hot spots for Raman tags. SERS is detected due to specific biochemical interactions such as antibody-antigen and DNA hybridization. Examples of specific biochemical interactions are antigen-antibody binding, nucleic acid hybridization, enzyme-substrate binding, ligand-receptor binding and etc. The term “event-specific Raman particle aggregation” means that Raman particles aggregate when a specific biochemical interaction is happening. When Raman particles aggregate, a unique electromagnetic field is generated between particles due to a dish-antenna type effect which enhance the Raman spectra of molecules located in the electromagnetic field resulting in the so-called Raman hot spots.

Other embodiments of the invention relate to a new methodology for SERS measurement with improved sensitivity, precision, and detection limits. This new approach also takes the advantage of the fact that the Raman signals of molecules at the junction of the metal particles are most enhanced electromagnetically.

The noble metal particles (e.g. silver and gold) are functionalized so that their mixing allows the analyte molecules to situate primarily in particle junction regions. For example, the “capture” particles can be coated with a layer of cationic polymer with suitable functional groups to promote adsorption of analyte molecules before the “capture” particles are mixed with excessive amounts of negatively charged silver nanoparticles. The capture particles can be further conjugated with antibodies or DNA so that they can form clusters with defined geometry with metal particles conjugated with SERS particles. Columbic interaction and specific interaction between biomolecules (e.g. biotin-streptavidin, antigen-antibody, and complimentary oligonucleotides) could be employed to create metal clusters to capture analyte molecules in the center of the clusters. The same approach can be also used to fabricate composite-inorganic-organic nanoparticles (COIN) with well defined geometry. COINS, as explained above, include clusters of noble metal nanoparticles with Raman active compounds embedded between the nanoparticles. If the analytes are Raman active molecules with strong intensity, the clusters formed during the Raman analysis could be COINS with well defined geometry.

To generate capture particles, the coinage metal nanoparticles can be modified in various ways to improve adsorption affinity of analyte molecules by taking advantage of one or more type of interactions including electrostatic, hydrophobic, covalent binding and specific interactions between the analyte molecules and the modified surface.

Electrostatic interaction: Silver and gold nanoparticles prepared by reduction of the metal ions with common reducing agents such as citrate and sodium borohydride have negative surface charges primarily due to the adsorption of major anions (citrate or BH₄⁻) in solution. Those negatively charged nanoparticles have strong affinity for most positively charged molecules as the strong electrostatic attraction brings the analyte molecules close to the particle surface. However, for negatively charged molecules, low SERS signal intensity is expected unless the electrostatic repulsion is overwhelmed by specific interactions between the molecules and the surface. To overcome this difficulty, the nanoparticles surface can be made to carry positive charges by adsorption of (1) simple cations (e,g Calcium and Ferric ions), (2) small molecules (e.g. thiol amines), (3) cationic polymers (e.g. polyallyamine and polyethyleneimine).

Hydrophobic interaction: Most of large organic molecules of medical and environmental interest are generally at least partially hydrophobic. This is one of main reasons for the wide applicability of reverse phase HPLC as an analytical tool. An organic coating can be created on silver/gold particles to retain various analyte molecules as in the case of reverse phase chromatography. For example, alkyl chains of different lengths (from C4 to C18) can be grafted to gold particles or gold coated silver particles.

In other embodiments of the invention, other functional groups can also be introduced in the organic phase to include more specific interactions such as H-bonding and ion-pairing. The strong S—Au interaction can also be employed to introduce various functional groups into the hydrophobic layer around the particles. For example, bifunctional molecules of the type HS—R—X can be adsorbed on gold or gold coated silver surface, where X can be carboxyl, amine, hydroxyl, and so on, and R can be any type of hydrocarbon moiety with or without various functional groups to facilitate specific interactions with the analyte molecules. For example, the introduction of amine or positively charged ammonium groups will facilitate electrostatic interaction with negatively charged analyte molecules. Similarly, the presence of negatively charged functional groups such sulfate and phosphate can facilitate ion-pairing with positively charged analytes.

Alternatively in other embodiments of the invention, the metal nanoparticles can be coated with a thin layer of silica and then various organic molecules can be attached through the silanol groups. One facile approach is to use silane compounds of the type, X—R—Si(OCH₃)₃ or X—R—Si(OCH₂CH₃), where R can be alkyl or any hydrocarbon moiety with or without functional groups, X can be H or various functional groups such as —COOH, —NH₂, —N(CH₃)₃⁺, etc. Organic solvent such as ethanol may used to dissolve the silane compounds before silica coated silver or gold particles are added.

Yet in other embodiments of the invention, layer by layer (LBL) adsorption of oppositely charged polymers can also be used to create a very stable organic layer with various functional groups from the polymers. For example, polyallylamine and polyacrylic acid can be applied alternatively to form the LBL coating. Other cationic and anionic polymers with suitable solubility can also be used to create multiply layers of polymer coating. Usually, two types of polymers, one cationic, the other anionic, are sufficient to create the coating of any desirable thickness. However, more types of polymers can be used so that the desired functional groups can be introduced at certain distance from the native metal surface.

Specific interaction: Covalent bonding and other strong specific interactions such as hydrogen bonding between complimentary oligonucleic acid strands as well as antibody-antigen interaction can be used to bring the analyte molecules very close to the native or derivatized metal particle surface. For example, when analyzing thiol-containing compounds, gold nanoparticles or silver particles with a thin gold layer can be used as a SERS substrate to take the advantage of the strong S—Au interaction.

In other embodiments, the SERS particles can also be coupled with certain reactive molecules or polymers which can form covalent bonds with analytes containing given functional groups. For example, there are several functional groups that can be used to attach an analyte to the particle surface. One such example is an epoxide. A polymer containing epoxide groups can be adsorbed on to the surface of the nanoparticle by adding a concentrated nanoparticle suspension to a large volume of the polymer solution. A good starting point is to add one volume of the nanoparticle suspension to four volumes of the polymer solution. The excess polymer is then removed by centrifugation and the modified particles are resuspended in the desired medium. An analyte containing amine groups can be reacted with this polymer at an alkaline pH resulting in the formation of a secondary amine bond.

In yet other embodiments of the invention relating to analyzing proteins or peptides, the capture particles can be derivatized with ligands or probes with strong affinity for the analytes, for instance, Glutathione for Glutathione-S-Transferase (GST)-proteins and boronic acid or lectin for glycoproteins and polysaccharides.

In one example, the nanoparticles can be modified by using thiol chemistry to attach a metal chelating group to the nanoparticle surface. A compound containing both a thiol group and a metal chelating group such as nitrilotriacetic acid (NTA) is dissolved in an appropriate solvent (e.g., water or ethanol) and the nanoparticles are then added to this solution. The resulting mixture is allowed to equilibrate for up to 12 hours, after which the nanoparticles are then washed by centrifugation. The desired metal is then added to the modified nanoparticles. For the capture of phosphopeptides or proteins, iron (III) is commonly used. The modified nanoparticles are suspended in a dilute solution of iron (III) chloride at acidic pH. After equilibration, the excess iron solution is removed by centrifugation and the iron-loaded particles are resuspended in an acidic buffer. The phosphopeptide is then introduced to the iron-loaded particles and allowed to equilibrate. The phosphate forms a complex with the iron on the nanoparticle surface and thus should be close enough to the metal surface to obtain a strong surface-enhanced Raman signal.

In other embodiments of the invention, the capture particles can also be first conjugated with the antigens/antibodies for a given protein to be analyzed. For example, antibodies can be conjugated to the nanoparticle surface using N-hydroxysuccinimide (NHS) chemistry. The nanoparticle surface is first modified by mixing with a compound containing a thiol group at one end and a carboxylic acid on the other. The compound attaches to the nanoparticle surface via the thiol group, leaving the carboxylic acid as the exposed functional group. This carboxylic acid is then converted to an NHS-ester, and resulting particles are washed and then mixed with the antibody. The amine groups of the antibody react with the NHS-ester to form an amide bond, thus creating antibody-labeled nanoparticles. Alternatively, a protein can be adsorbed onto the nanoparticle surface and then used to bind the antibody of choice. This prevents the need for developing a separate nanoparticle for each specific antibody.

The embodiments of the invention include: (a) Design and fabrication of Raman particles with tag molecules for SERS and with probes for specific biochemical bindings. (b) SERS detection of target molecules using laser excitation on tags upon particle aggregation caused by specific probe-target bindings. (c) Methods for preparing capture particles (for analyte absorption) and enhancer particles (which surround the capture particle) by using specific interactions. (d) SERS measurement sequence including (1) incubating the capture particles with analytes; and (2) addition of enhancer particles to form clusters for improved Raman scattering intensity.

The embodiments of this invention addresses the problem of: (1) Target molecule detection at single molecule level. (2) Highly sensitive detection of antigens, antibodies, and viruses. (3) Genetic detection of nucleic acids without the use of Polymerase Chain Reaction (PCR) amplification. As a result, the embodiments of the invention could simplify the sample preparation and lower the cost significantly.

In the embodiments of the invention, silver and gold colloidal particles are preferred substrate for SERS. However, typically, few analyte molecules on the particle surface have extremely high enhanced signals (over 10¹⁰). Applicants have recognized that these molecules are likely to be located in the gaps between two nearly touching nanoparticles. Theoretical calculation shows that when the gap is less than 5 nm, strong electromagnetic enhancement occurs. The embodiments of the invention include a new scheme to generate clusters of the metal particles with the analyte molecules enriched at the particle junctions to improve the detection limit by SERS. Moreover, the metal cluster size could be better controlled by the proposed measurement scheme so that quantitative aspect of SERS can be improved.

Some of the technical advantages of the embodiments of the invention relate to the following:

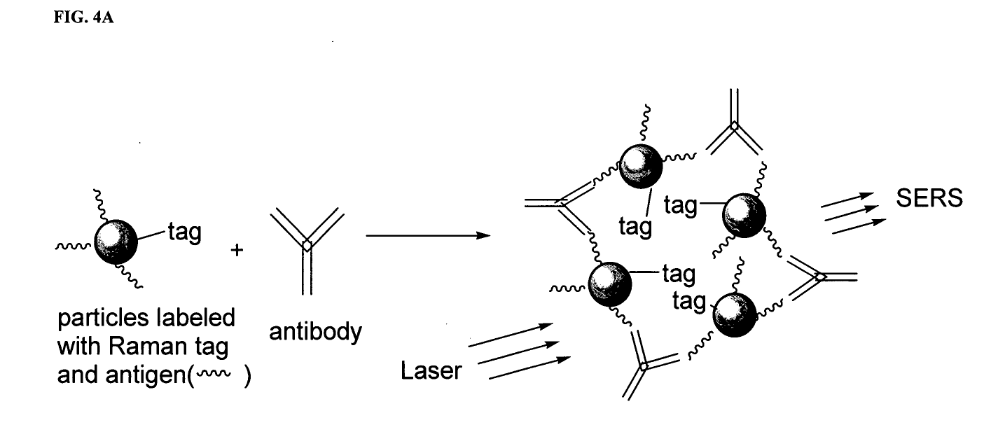

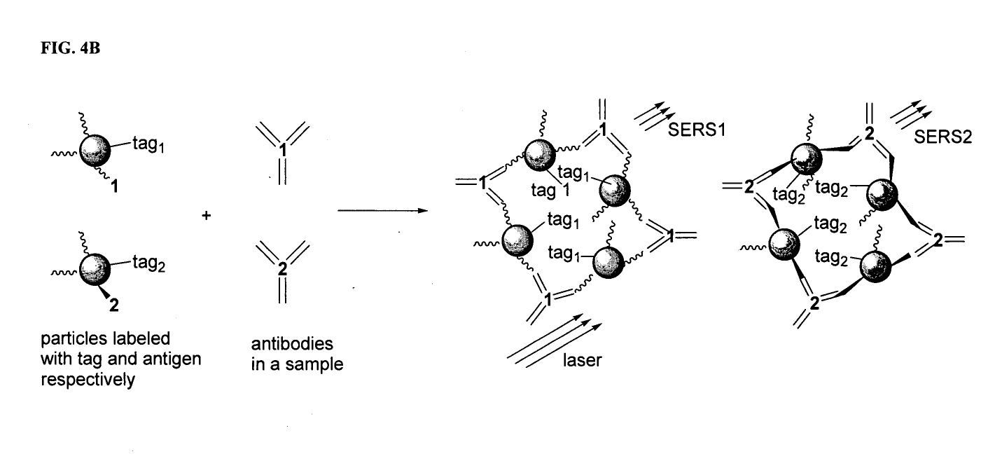

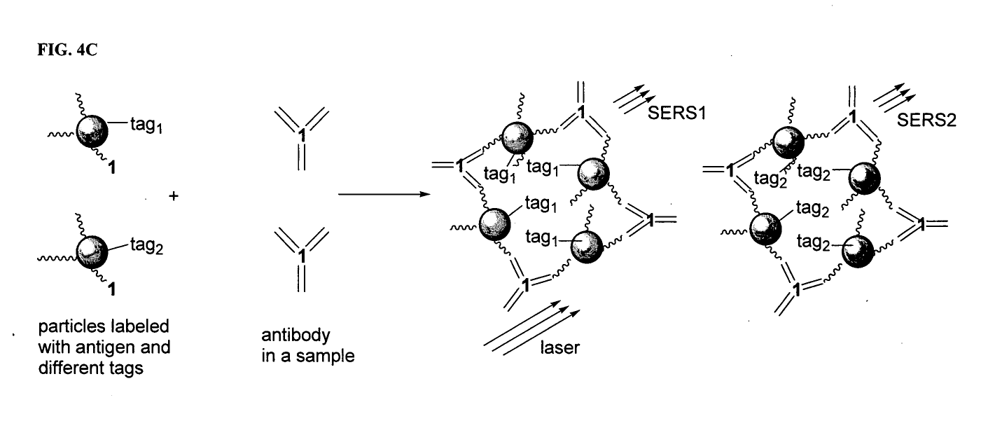

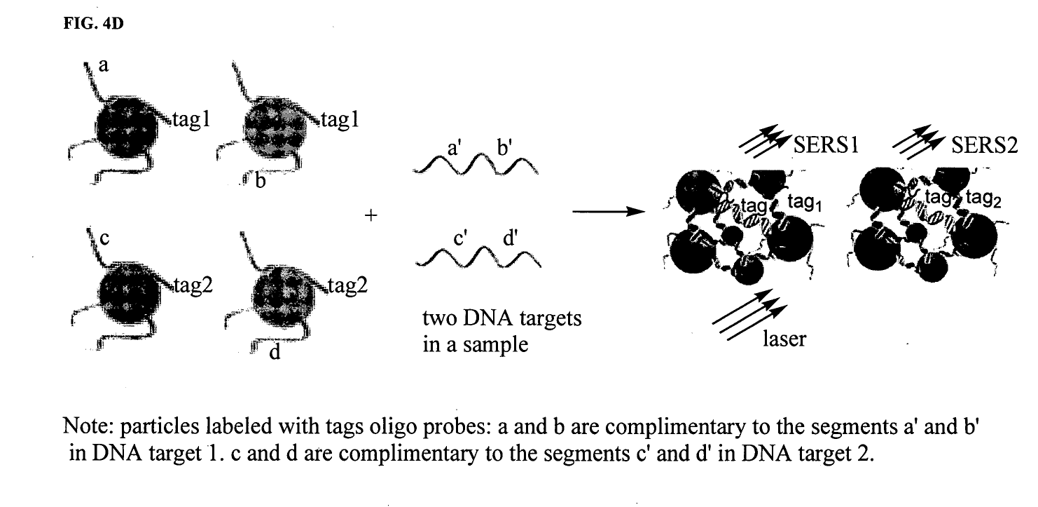

(1) Target samples (proteins or nucleic acids) need not be labeled (proteins or antibodies) or amplified (DNA). This is because SERS detection could be a label free strategy that is superior to fluorescence technology in which target sample has to be labeled with fluorescent molecules. FIG. 4A demonstrates this advantage with reference to an antigen-antibody specific SERS measurement. Thus tedious, error-prone, and costly sample preparation steps can be avoided.
(2) Multiple targets can be detected at the same time in one sample by using multiple Raman particles with different Raman tags and detection probes. FIG. 4B demonstrates this advantage with reference to a SERS measurement of multiplexed assay containing Raman particles with different Raman tags and detection probes.
(3) The detection of each target molecule can be verified by duplication using the same detection probe but with a different Raman tag. The multiplicity of Raman tagging strategy also allows redundant measurement, thereby providing greater control increased credibility of the test results. FIG. 4C demonstrates this advantage.

### (4) The structure of Raman tagged and probe attached particles can be easily fine tuned to meet specific applications in diagnostics and drug discovery.

### (5) The embodiments of the invention could allow clusters of well defined size and shape, optimized for SERS effects, to be prepared.

(6) The embodiments of the invention could allow the analyte molecules to be concentrated in the gaps between neighboring particles, markedly increasing the Raman scattering intensity. The SERS measurement scheme according to the embodiments of the invention ensures that the analyte molecules could be between particles. The reasons for increased Raman intensity is possibly due to an electromagnetic effect and a “chemical” effect, resulting in a resonance Raman enhancement effect of specific metal-molecule complexes. For example, the electromagnetic effect could produce enhancement of the order of 10¹¹-10¹³ while the “chemical” SERS effect could a further enhancement to raise the total enhancement to an order of about 10¹⁴-10¹⁸.

Raman-Active Particles (SERS Particles)

The Raman active particle is provided by metal nanoparticles, which may used alone or in combination with other Raman active particles, such as a metal-coated porous silicon substrate to further enhance the Raman signal obtained from small numbers of molecules of a sample such as an analyte. In various embodiments of the invention, the nanoparticles are silver, gold, platinum, copper, aluminum, or other conductive materials, although any nanoparticles capable of reflecting a Raman signal may be used. Particles made of silver or gold are especially preferred.

The particles or colloid surfaces can be of various shapes and sizes. In various embodiments of the invention, nanoparticles of between 1 nanometer (nm) and 2 micrometers (micron) in diameter may be used. In alternative embodiments of the invention, nanoparticles of 2 nm to 1 micron, 5 nm to 500 nm, 10 nm to 200 nm, 20 nm to 100 nm, 30 nm to 80 nm, 40 nm to 70 nm or 50 nm to 60 nm diameter may be used. In certain embodiments of the invention, nanoparticles with an average diameter of 10 to 50 nm, 50 to 100 nm or about 100 nm may be used. If used in combination with other Raman active particles, such as a metal-coated porous silicon substrate, the size of the nanoparticles could depend on the other surface used. For example, the diameter of the pores in the metal-coated porous silicon may be selected so that the nanoparticles fit inside the pores.

The nanoparticles may be approximately spherical, cylindrical, triangular, rod-like, edgy, multi-faceted, prism, or pointy in shape, although nanoparticles of any regular or irregular shape may be used. In certain embodiments of the invention, the nanoparticles may be single nanoparticles, and/or random aggregates of nanoparticles. The aggregates can be synthesized by standard techniques, such as by adding electrolytes, such as NaCl, to the nanoparticle suspension. The aggregation can be induced by addition of polymeric substance, especially polyelectrolytes with opposite charges to the colloidal particles. It is also possible to induce colloidal aggregation by “depletion mechanism,” wherein the addition of non-adsorbing polymers effectively results in an attraction potential due to the depletion of the polymer molecules from the region between two closely approaching nanoparticles.

Nanoparticles may be cross-linked to produce particular aggregates of nanoparticles, such as dimers, trimers, tetramers or other aggregates. Formation of “hot spots” may be associated with particular aggregates or colloids (optionally with ionic compounds) of nanoparticles. Certain embodiments of the invention may use heterogeneous mixtures of aggregates or colloids of different size, while other embodiments may use homogenous populations of nanoparticles and/or aggregates or colloids (optionally with ionic compounds). In certain embodiments of the invention, aggregates containing a selected number of nanoparticles (e.g., dimers, trimers, etc.) may be enriched or purified by known techniques, such as ultracentrifugation in sucrose gradient solutions. In various embodiments of the invention, nanoparticle aggregates or colloids (optionally with ionic compounds) of about 5, 10, 20, 40, 60, 80, 100, 200, 300, 400, 500, 600, 700, 800, 900 to 1000 nm in size or larger are used. In particular embodiments of the invention, nanoparticle aggregates (optionally produced with addition of ionic compounds) may be between about 10 nm and about 200 nm in size.

The nanoparticles may be crosslinked to form aggregates by techniques known in the art. For example, gold nanoparticles may be cross-linked, for example, using bifunctional linker compounds bearing terminal thiol or sulfhydryl groups. In some embodiments of the invention, a single linker compound may be derivatized with thiol groups at both ends. Upon reaction with gold nanoparticles, the linker could form nanoparticle dimers that are separated by the length of the linker. In other embodiments of the invention, linkers with three, four or more thiol groups may be used to simultaneously attach to multiple nanoparticles. The use of an excess of nanoparticles to linker compounds prevents formation of multiple cross-links and nanoparticle precipitation. Aggregates of silver nanoparticles may also be formed by standard synthesis methods known in the art.

The nanoparticles and their aggregates may be covalently attached to a molecular sample such as an analyte. In alternative embodiments of the invention, the molecular sample may be directly attached to the nanoparticles, or may be attached to linker compounds that are covalently or non-covalently bonded to the nanoparticles aggregates.

It is contemplated that the linker compounds used to attach molecule(s) of the sample such as an analyte may be of almost any length, ranging from about 0.05, 0.1, 0.2, 0.5, 0.75, 1, 2, 3, 4, 5, 6, 7, 8, 9, 10, 11, 12, 13, 14, 15, 16, 17, 18, 19, 20, 21, 22, 23, 24, 25, 27, 30, 35, 40, 45, 50, 55, 60, 65, 60, 80, 90 to 100 nm or even greater length. Certain embodiments of the invention may use linkers of heterogeneous length.

The molecule(s) of the sample such as an analyte may be attached to nanoparticles as they travel down a channel to form molecular-nanoparticle complex. In certain embodiments of the invention, the length of time available for the cross-linking reaction to occur may be very limited. Such embodiments may utilize highly reactive cross-linking groups with rapid reaction rates, such as epoxide groups, azido groups, arylazido groups, triazine groups or diazo groups. In certain embodiments of the invention, the cross-linking groups may be photoactivated by exposure to intense light, such as a laser. For example, photoactivation of diazo or azido compounds results in the formation, respectively, of highly reactive carbene and nitrene moieties. In certain embodiments of the invention, the reactive groups may be selected so that they can attach the nanoparticles to a sample such as an analyte, rather than cross-linking the nanoparticles to each other. The selection and preparation of reactive cross-linking groups capable of binding to a sample such as an analyte is known in the art. In alternative embodiments of the invention, components such as analytes may themselves be covalently modified, for example with a sulfhydryl group that can attach to gold nanoparticles.

The nanoparticles or other Raman active particles may be coated with derivatized silanes, such as aminosilane, 3-glycidoxypropyltrimethoxysilane (GOP) or aminopropyltrimethoxysilane (APTMS). The reactive groups at the ends of the silanes may be used to form cross-linked aggregates of nanoparticles. It is contemplated that the linker compounds used may be of almost any length, ranging from about 0.05, 0.1, 0.2, 0.5, 0.75, 1, 2, 3, 4, 5, 6, 7, 8, 9, 10, 11, 12, 13, 14, 15, 16, 17, 18, 19, 20, 21, 21, 22, 23, 24, 25, 27, 30, 35, 40, 45, 50, 55, 60, 65, 70, 80, 90, to 100 nm or even greater length.

The nanoparticles may be modified to contain various reactive groups before they are attached to linker compounds. Modified nanoparticles are commercially available, such as the Nanogold® nanoparticles from Nanoprobes, Inc. (Yaphank, N.Y.). Nanogold® nanoparticles may be obtained with single or multiple maleimide, amine or other groups attached per nanoparticle. The Nanogold® nanoparticles are also available in either positively or negatively charged form to facilitate manipulation of nanoparticles in an electric field. Such modified nanoparticles may be attached to a variety of known linker compounds to provide dimers, trimers or other aggregates of nanoparticles.

The type of linker compound used is not limiting. In some embodiments of the invention, the linker group may comprise phenylacetylene polymers. Alternatively, linker groups may comprise polytetrafluoroethylene, polyvinyl pyrrolidone, polystyrene, polypropylene, polyacrylamide, polyethylene or other known polymers. The linker compounds of use are not limited to polymers, but may also include other types of molecules such as silanes, alkanes, derivatized silanes or derivatized alkanes. In particular embodiments of the invention, linker compounds of relatively simple chemical structure, such as alkanes or silanes, may be used to avoid interfering with the Raman signals emitted by a sample such as an analyte.

Alternatively, the linker compounds used may contain a single reactive group, such as a thiol group. Nanoparticles containing a single attached linker compound may self-aggregate into dimers, for example, by non-covalent interaction of linker compounds attached to two different nanoparticles. For example, the linker compounds may comprise alkane thiols. Following attachment of the thiol group to gold nanoparticles, the alkane groups could tend to associate by hydrophobic interaction. In other alternative embodiments of the invention, the linker compounds may contain different functional groups at either end. For example, a liker compound could contain a sulfhydryl group at one end to allow attachment to gold nanoparticles, and a different reactive group at the other end to allow attachment to other linker compounds. Many such reactive groups are known in the art and may be used in the present methods and apparatus.

In other embodiments of the invention, a sample such as an analyte is closely associated with the surface of the nanoparticles or may be otherwise in close proximity to the nanoparticles (between about 0.2 and 1.0 nm). As used herein, the term “closely associated with” refers to a molecular sample such as an analyte which is attached (either covalent or non-covalent) or adsorbed on a Raman-active surface. The skilled artisan could realize that covalent attachment of a molecular sample such as an analyte to nanoparticles is not required in order to generate a surface-enhanced Raman signal.

To facilitate detection of a sample such as an analyte, one embodiment of the invention comprises materials that are transparent to electromagnetic radiation at the excitation and emission frequencies used. Glass, silicon, quartz, or any other materials that are generally transparent in the frequency ranges used for Raman spectroscopy may be used. Any geometry, shape, and size is possible for the sample stage since any refraction which this component introduces can be ignored or compensated for.

Raman Labels (Tags)

Certain embodiments of the invention may involve attaching a label to one or more molecules of a sample such as an analyte to facilitate their measurement by the Raman detection unit. Non-limiting examples of labels that could be used for Raman spectroscopy include TRIT (tetramethyl rhodamine isothiol), NBD (7-nitrobenz-2-oxa-1,3-diazole), Texas Red dye, phthalic acid, terephthalic acid, isophthalic acid, cresyl fast violet, cresyl blue violet, brilliant cresyl blue, para-aminobenzoic acid, erythrosine, biotin, digoxigenin, 5-carboxy-4′,5′-dichloro-2′,7′-dimethoxy fluorescein, 5-carboxy-2′,4′,5′,7′-tetrachlorofluorescein, 5-carboxyfluorescein, 5-carboxy rhodamine, 6-carboxyrhodamine, 6-carboxyletramethyl amino phthalocyanines, azomethines, cyanines, xanthines, succinylfluoresceins, aminoacridine, quantum dots, carbon nanotubes, fullerenes, organocyanides, such as isocyanide, and the like.

Polycyclic aromatic compounds may function as Raman labels, as is known in the art. Other labels that may be of use for particular embodiments of the invention include cyanide, thiol, chlorine, bromine, methyl, phosphorus and sulfur. The Raman labels used should generate distinguishable Raman spectra and may be specifically bound to or associated with different types of samples such as analytes.

Labels may be attached directly to the molecule(s) of a sample such as an analyte or may be attached via various linker compounds. Cross-linking reagents and linker compounds of use in the disclosed methods are further described below.

Applications of the Embodiments of the Invention

The applications of the embodiment of this invention include material inspection, biologic cell or tissue imaging, and in vivo imaging, particularly of a sample obtained from a biological source. The sample could be a biological cell or tissue. For example, the sample could be a phosphorylated peptide. In this case, by the embodiments of this invention, the user can detect the position and spatial location of phosphorylation within the sample by either systematically moving the sample stage or steering the beam through the body of the sample. Also, by the embodiments of this invention, the user can do imaging of multiple layers of tissues, for example.

Typically, a sample obtained from a biologic source, such as, for example, a bodily fluid or cell lysate solution, is a complex mixture of proteins and other molecules. The components of the mixture can be separated using known techniques for isolating protein fractions from biologic samples, such as, for example, physical or affinity based separation techniques. The isolated proteinaceous fraction can then be digested into smaller peptides. Typical methods include enzymatic digestions such as, for example, proteinase enzymes such as, Arg-C(N-acetyl-gamma-glutamyl-phosphate reductase), Asp-N, Glu-C, Lys-C, chromotrypsin, clostripain, trypsin, and thermolysin. The resulting digest of peptides can be further separated, for example, using HPLC (high performance liquid chromatography). Raman spectroscopy can then be performed on the resulting sample by, for example, mixing the digested sample with a SERS solution, such as, for example, a colloidal silver solution, depositing and drying the digested sample onto a substrate and subsequently adding a SERS solution, such as a colloidal silver solution, depositing the sample onto a SERS-active substrate, or it can be performed in-line in a component of a microfluidic or nanofluidic system, such as by using a micro or nanomixer to mix the SERS solution with a the digested sample and subsequently performing Raman analysis on the sample. A silver colloidal solution can be mixed with digested sample eluants in a fluidic format (optionally, on a chip) and the detection can be performed inline as the eluants are flowing through the laser detection volume. In additional embodiments, some or all of these steps are performed using microfluidics.

For biological imaging of cells or tissue by the embodiments of this invention, the cell or tissue to be analyzed could be stained with metallic nanoparticles. The metallic nanoparticles may settle on the cell or tissue surface, or may bind to specific molecules in the cell or tissue, if the nanoparticles are coated with antibodies. Alternatively, the nanoparticles may contain signaling molecules (e.g. composite organic-inorganic nanoparticles (COIN) or other SERS labels).

In another embodiment the SERS array could include surface enhanced Raman scattering active particles that do not contain Raman-labels. For example, gold silver, platinum copper or aluminum particles can be placed in the array to enhance the Raman spectra of Raman active analytes. Silver colloidal particles have been found to be particularly useful for SERS arrays. Since these SERS active particles do not themselves produce the detected Raman spectra, the sample such as an analyte must produce a detectable Raman Spectra. However, surface enhanced Raman scattering (SERS) techniques make it possible to obtain many-fold Raman signal enhancement, for example, by about 10 to about 10000 fold increase, more preferably, about 100 to about 1000 fold increase. Such huge enhancement factors could be attributed primarily to enhanced electromagnetic fields on curved surfaces of coinage metals. Although the electromagnetic enhancement (EME) has been shown to be related to the roughness of metal surfaces or particle size when individual metal colloids are used, SERS is most effectively detected from aggregated colloids. For example, chemical enhancement can also be obtained by placing molecules in a close proximity to the surface in certain orientations.

The Raman particle platforms of the embodiments of the invention could be built using technology developed by Applicants. For example, probes can be attached to COINs through adsorption of the probe onto the COIN surface. Alternatively, COINs may be coupled with probes through biotin-avidin linkages. For example, avidin or streptavidin (or an analog thereof) can be adsorbed to the surface of the COIN and a biotin-modified probe contacted with the avidin or streptavidin-modified surface forming a biotin-avidin (or biotin-streptavidin) linkage. Optionally, avidin or streptavidin may be adsorbed in combination with another protein, such as BSA, and optionally be crosslinked. In addition, for COINs having a functional layer that includes a carboxylic acid or amine functional group, probes having a corresponding amine or carboxylic acid functional group can be attached through water-soluble carbodiimide coupling reagents, such as EDC (1-ethyl-3-(3-dimethyl aminopropyl)carbodiimide), which couples carboxylic acid functional groups with amine groups.

Furthermore, the applicants have built a state of the art Raman spectrometry facility and developed techniques to perform the SERS detection. The applicants have experimentally demonstrated that a single deoxyadenosine monophosphate (“dAMP”) molecule can be detected with a Raman system.

While studying biological molecules, the applicants observed that SERS enhancement is significantly affected by introducing particular cations to the SERS sample. The applicants screened multiple chemical salts for nucleotides, nucleosides, bases, and dye molecules, and observed that cations affect the SERS signal enhancement for all the molecules Applicants investigated. Among the salts screened, lithium chloride (LiCl) generated the strongest SERS enhancement, even stronger than other commonly used chemical enhancers, such as sodium chloride (NaCl) and potassium chloride (KCl). This strong enhancement with LiCl was observed for various target molecule concentrations, ranging from sub-monolayer coverage of the silver colloid surface to saturated surface coverage.

In order to study the source of this enhancement, various SERS measurements were performed with multiple ionic salts. Different excitation wavelengths were investigated to determine the contribution of electromagnetic enhancement. Also, target molecules with different charges were tested to study the electrostatic interaction between the target molecules and the metal nanoparticles. In addition, metallic and nanostructured substrates were compared with colloidal nanoparticles to determine whether colloidal aggregation is a factor for this enhancement. In every case tested, strong SERS enhancement with LiCl was observed.

The applicants took advantage of this substantial increase in SERS enhancements to perform rapid and reliable detection at single molecule concentrations. The diffusion of adenine and dAMP molecule was monitored, utilizing SERS with LiCl at a fraction of a second collection time. These results demonstrated that the selection of cation/anion combination as a chemical enhancer greatly assists the ultra-sensitive detection of molecules.

### EXAMPLES

### Example 1

### Preparation of Silver Colloids

To a 250 mL round bottom flask equipped with a stirring bar, was added 100 mL de-ionized water and 0.200 mL of a 0.500 M silver nitrate solution. The flask was shaken to thoroughly mix the solution. 0.136 mL of a 0.500 M sodium citrate solution was then added to the flask using a 200 μl pipette. The flask was then placed in a heating mantle and the stirrer was set at medium speed. A water cooled condenser was attached to the flask and heating commenced. The heating mantle was applied at maximum voltage, resulting in boiling of the solution between 7 and 10 minutes. Color changes occur within 120 seconds of boiling. The heating is stopped after 60 minutes, the solution is cooled to room temperature and the resulting colloidal suspension is transferred to a 100 mL glass bottle for storage.

### Example 2

### COIN Synthesis

In general, Raman labels were pipetted into the COIN synthesis solution to yield final concentrations of the labels in synthesis solution of about 1 to about 50 micromole. In some cases, acid or organic solvents were used to enhance label solubility. For example, 8-aza-adenine and N-benzoyladenine were pipetted into the COIN formation reaction as 1.00 mM solutions in 1 mM HCl, 2-mercapto-benzimidazole was added from a 1.0 mM solution in ethanol, and 4-amino-pyrazolo[3,4-d]pyrim-idine and zeatin were added from a 0.25 mM solution in 1 mM HNO₃.

Reflux Method: To prepare COIN particles with silver seeds, typically, 50 mL silver seed suspension (equivalent to 2.0 mM Ag⁺) was heated to boiling in a reflux system before introducing Raman labels. Silver nitrate stock solution (0.50 M) was then added dropwise or in small aliquots (50-100 microliter) to induce the growth and aggregation of silver seed particles. Up to a total of 2.5 mM silver nitrate could be added. The solution was kept boiling until the suspension became very turbid and dark brown in color. At this point, the temperature was lowered quickly by transferring the colloid solution into a glass bottle. The solution was then stored at room temperature. The optimum heating time depended on the nature of Raman labels and amounts of silver nitrate added. It was found helpful to verify that particles had reached a desired size range (80-100 nm on average) by PCS or UV-Vis spectroscopy before the heating was arrested. Normally, the dark brown color was an indication of cluster formation and associated Raman activity.

To prepare COIN particles with gold seeds, typically, gold seeds were first prepared from 0.25 mM HAuCl₄ in the presence of a Raman label (for example, 20 micromole 8-aza-adenine). After heating the gold seed solution to boiling, silver nitrate and sodium citrate stock solutions (0.50 M) were added, separately, so that the final gold suspension contained 1.0 mM AgNO₃ and 1.0 mM sodium citrate. Silver chloride precipitate might form immediately after silver nitrate addition but disappeared soon with heating. After boiling, an orange-brown color developed and stabilized. An additional aliquot (50-100 microliter) of silver nitrate and sodium citrate stock solutions (0.50 M each) was added to induce the development of a green color, which was the indication of cluster formation and was associated with Raman activity.

Note that the two procedures produced COINs with different colors, primarily due to differences in the size of primary particles before cluster formation.

Oven Method: COINs could also be prepared conveniently by using a convection oven. Silver seed suspension was mixed with sodium citrate and silver nitrate solutions in a 20 mL glass vial. The final volume of the mixture was typically 10 mL, which contained silver particles (equivalent to 0.5 mM Ag⁺), 1.0 mM silver nitrate and 2.0 mM sodium citrate (including the portion from the seed suspension). The glass vials were incubated in the oven, set at 95.degree. C., for 60 min before being stored at room temperature. A range of label concentrations could be tested at the same time. Batches showing brownish color with turbidity were tested for Raman activity and colloidal stability. Batches with significant sedimentation (which occurred when the label concentrations were too high) were discarded. Occasionally, batches that did not show sufficient turbidity could be kept at room temperature for an extended period of time (up to 3 days) to allow cluster formation. In many cases, suspensions became more turbid over time due to aggregation, and strong Raman activity developed within 24 hours. A stabilizing agent, such as bovine serum albumin (BSA), could be used to stop the aggregation and stabilize the COIN particles.

A similar approach was used to prepare COINs with gold cores. Briefly, 3 mL of gold suspensions (0.50 mM Au³⁺) prepared in the presence of Raman labels was mixed with 7 mL of silver citrate solution (containing 5.0 mM silver nitrate and 5.0 mM sodium citrate before mixing) in a 20 mL glass vial. The vial was placed in a convection oven and heated to 95.degree. C. for 1 hour. Different concentrations of labeled gold seeds could be used simultaneously in order to produce batches with sufficient Raman activities. It should be noted that a COIN sample can be heterogeneous in terms of size and Raman activity. We typically used centrifugation (200-2,000.times.g for 5-10 min) or filtration (300 kDa, 1000 kDa, or 0.2 micron filters, Pall Life Sciences through VWR) to enrich for particles in the range of 50-100 nm. It is recommended to coat the COIN particles with a protection agent (for example, BSA, antibody) before enrichment. Some lots of COINs that we prepared (with no further treatment after synthesis) were stable for more than 3 months at room temperature without noticeable changes in physical and chemical properties.

Cold Method: 100 mL of silver particles (1 mM silver atoms) were mixed with 1 mL of Raman label solution (typically 1 mM). Then 5 to 10 mL of 0.5 M LiCl solution was added to induce silver aggregation. As soon as the suspension became visibly darker (due to aggregation), 0.5% bovine serum albumin (BSA) was added to inhibit the aggregation process. Afterwards, the suspension was centrifuged at 4500 g for 15 minutes. After removing the supernatant (mostly single particles), the pellet was resuspended in 1 mM sodium citrate solution. The washing procedure was repeated for a total of three times. After the last washing, the resuspended pellets were filtered through 0.2 micromole membrane filter to remove large aggregates. The filtrate was collected as COIN suspension. The concentrations of COINs were adjusted to 1.0 or 1.5 mM with 1 mM sodium citrate by comparing the absorbance at 400 nm with 1 mM silver colloids for SERS.

Coating Particles with BSA: COIN particles were coated with an adsorption layer of BSA by adding 0.2% BSA to the COIN synthesis solution when the desired COIN size was reached. The addition of BSA inhibited further aggregation.

Crosslinking the BSA Coating: The BSA adsorption layer was crosslinked with glutaraldehyde followed by reduction with NaBH₄. Crosslinking was accomplished by transferring 12 mL of BSA coated COINs (having a silver concentration of about 1.5 mM) into a 15 mL centrifuge tube and adding 0.36 g of 70% glutaraldehyde and 213 microliter of 1 mM sodium citrate. The solution was mixed well and allowed to sit at room temperature for about 10 min. before it was placed in a refrigerator at 4.degree. C. The solution remained at 4.degree. C. for at least 4 hours and then 275 microliter of freshly prepared NaBH₄ (1 M) was added. The solution was mixed and left at room temperature for 30 min. The solution was then centrifuged at 5000 rpm for 60 min. The supernatant was removed with a pipette, leaving about 1.2 mL of liquid and the pellet in the centrifuge tube. 0.8 mL of 1 mM sodium citrate was added to yield a final volume of 2.0 mL. The coated COINs were purified by FPLC size-exclusion chromatography on a crosslinked agarose column.

Particle Size Measurement: The sizes of silver and gold seed particles as well as COINs were determined by using Photon Correlation Spectroscopy (PCS, Zetasizer3 3000 HS or Nano-ZS, Malvern). All measurements were conducted at 25.degree. C. using a He—Ne laser at 633 nm. Samples were diluted with deionized water when necessary.

Raman Spectral Analysis: for all SERS and COIN assays in solution, a Raman microscope (Renishaw, UK) equipped with a 514 nm Argon ion laser (25 mW) was used. Typically, a drop (50-200 microliter) of a sample was placed on an aluminum surface. The laser beam was focused on the top surface of the sample meniscus and photons were collected for 10-20 second. The Raman system normally generated about 600 counts from methanol at 1040 cm⁻¹ for 10 second collection time. For Raman spectroscopy detection of analyte immobilized on surface, Raman spectra were recorded using a Raman microscope built in-house. This Raman microscope consisted of a water cooled Argon ion laser operating in continuous-wave mode, a dichroic reflector, a holographic notch filter, a Czerny-Turner spectrometer, and a liquid nitrogen cooled CCD (charge-coupled device) camera. The spectroscopy components were coupled with a microscope so that the microscope objective focused the laser beam onto a sample, and collected the back-scattered Raman emission. The laser power at the sample was .about.60 mW. All Raman spectra were collected with 514 nm excitation wavelength.

### Example 3

### Generation of Positively Charged Silver by Adsorption of Cationic Polymers

Applicants found that poly(2-metacryloxyethyltrimethylammonium bromide) (PQA) can reverse the surface charge of silver colloids at very low polymer concentration (1 ppm). At even higher polymer concentration, 50 ppm, the polymer coated silver has a zeta-potential as high as 50 mV. A stable positively charged silver suspension was prepared at concentrations (1 mM Ag). It was found that mixing of equal volumes of silver colloids and PQA led to aggregation. However, a stable suspension was obtained when a small volume of silver colloids was added into a larger volume of polymer solution. Thus, silver colloids were first concentrated 5 times by centrifugation (7500 rpm for 10 min). Then 1 volume of the concentrated silver sol was rapidly distributed into 4 volumes of polymer solution. The final suspension contained 1 mM silver (about 5.7×10¹⁰ particles/mL) and 800 ppm PQA (about 1.2×10¹³ chains/mL). Zeta potential measurements confirmed that the colloids were positively charged and PCS size measurement did not reveal significant change in particle size compared with the original silver colloids, suggesting that there was no significant aggregation during mixing. Applicants also demonstrated that polyethyleneimine (PEI) could also stabilize 24 nm Ag particles.

### Example 4

### Functionalized Silver Particles with Antibodies

First, Applicants encapsulated COIN with bovine serum albumin (BSA) by glutaraldehyde cross-linking, and then conjugated the BSA-encapsulated COIN with antibody molecules by EDC cross linking reaction. Alternatively direct adsorption antibody molecules on bare COIN surface could also be used. Applicants estimated that on average 50 antibody molecules could be attached to a single COIN particles. Applicants used Xenobind™ Aldehyde slides (Xenopore Inc., NJ, USA) as substrates for capture antibody immobilization. To immobilize capture antibodies, 50 μL of an antibody (9 μg/mL) in 0.33×PBS was added to each well that was 5 mm in diameter and 1 mm in depth, formed with a piece of cured poly(dimethyl siloxane) (PDMS) elastomer. Antigen binding and detection antibody binding (or antibody-COIN conjugate binding) were carried out following instructions from the antibody supplier (BD Biosciences).

Biological reagents including anti-IL-2 and anti-IL-8 antibodies were purchased from BD Biosciences Inc (San Jose, Calif., USA). The capture antibodies were monoclonal antibodies generated from mouse, and the detection antibodies were polyclonal antibodies generated from mouse and conjugated with biotin. Salt solutions and buffers were purchased from Ambion, Inc. (Austin, Tex., USA), including 5 M NaCl, 10×PBS (1×PBS 137 mM NaCl, 2.7 mM KCl, 8 mM Na₂HPO₄, and 2 mM KH₂PO₄, pH 7.4). Unless otherwise indicated, all other chemicals were purchased, at highest available quality, from Sigma Aldrich Chemical Company (St. Louis, Mo., USA). Deionized water (DI-water) used for all experiments had a resistance of 18.2×10⁶ Ohms-cm that was obtained with a water purification unit (Nanopure Infinity, Barnstead, USA).

In one embodiment of the invention, the conjugation of COIN particles with antibodies was undertaken as follows: A 500-μL solution containing 2 ng of a biotinylated anti-human IL-2 or IL-8 antibody (anti-IL-2 or anti-IL-8) in 1 mM sodium citrate (pH 9) was mixed with 500 μL of a COIN solution (made with 8-aza-adenine or N-benzoyl-adenine); the resulting solution was incubated at room temperature for 1 hour, followed by adding 100 μL of PEG-400 (polyethylene-glycol-400). The solution was incubated at room temperature for another 30 min before 200 μL of 1% Tween-20 was added. The solution was centrifuged at 2000×g for 10 min. After removing the supernatant, the pellet was resuspended in 1 mL solution containing 0.5% BSA, 0.1% Tween-20 and 1 mM sodium citrate (BSAT). The solution was then centrifuged at 1000×g for 10 min. The B SAT washing procedure was repeated for a total of 3 times. The final pellet was resuspended in 700 μL of diluting solution (0.5% BSA, 1×PBS, 0.05% Tween-20). The Raman activity of COIN was measured and adjusted to a specific activity of about 500 photon counts per μL per 10 seconds using a Raman microscope that generated about 600 counts from methanol at 1040 cm⁻¹ for 10 second collection time.

The confirmation of antibody-COIN conjugation made as follows: To obtain a standard curve, ELISA (enzyme-linked immunosorbent assay) experiments were performed according to manufacture's instruction (BD Bioscences), using immobilized capture antibody, a fixed analyte concentration (5 ng/mL IL-2 protein) and a serially diluted detection antibody (0, 0.01, 0.1, 1, and 10 μg/mL). After detection antibody binding, streptavidin-HRP (Horse Radish Peroxidase) was then reacted with the biotinylated detection antibodies and TMB (Tetramethyl Benzidine) substrate was applied followed by UV absorption measurement. Standard curve was generated by plotting absorption values against antibody concentrations. To estimate the amount of antibody molecules that could be attached to a COIN particle, similar ELISA experiment was then performed with COIN conjugated with a detection antibody. The ELISA data were collected and the binding activity of the COIN-antibody conjugate was compared with the standard curve to estimate the equivalent amount of antibody in the COIN-antibody conjugate, assuming that one of the antibody molecules that had been conjugated to a COIN particle bound to an immobilized analyte, and that all biotin moieties associated with the COIN particle were bound by streptavidin-HRP. Finally, the number of antibody molecules per COIN was estimated by dividing the equivalent amount of antibody in the COIN-antibody by the estimated number of COIN particle. Applicants estimated that there are approximately 50 antibody molecules on a COIN particle. The above procedures used for functionization of COINs can be used to introduce antibody, antigen, etc to “capture” and “enhancer” particles. They can also be used to functionize nanoparticles with different tags.

### Example 5

### Detection at Single Molecule Concentration

The experiments to perform rapid and reliable detection at single molecule concentrations were as follows. Raman spectra were recorded using an in-house built custom Raman microscope, unless specified otherwise. This Raman microscope consisted of a tunable titanium-doped sapphire laser operating in continuous-wave mode, a dichroic reflector, a holographic notch filter, a grating spectrometer, and a liquid nitrogen cooled CCD camera. The spectroscopy components were coupled with a microscope so that the microscope objective focused the laser beam onto a sample, and collected the back-scattered Raman emission. The laser power at the sample was ˜300 mW, and the beam diameter was approximately 5 μm. All Raman spectra were collected with 785 nm excitation wavelength, unless noted otherwise. This near-infrared excitation wavelength was chosen to avoid the electron resonance of the target molecules and hence signal increase or photodegradation of molecules associated with the resonance effect were minimized. Some spectra were collected by tuning the excitation wavelength to 830 nm and replacing several optical components. Spectra with 514 nm excitation were collected using a similarly configured in-house built Raman microscope with an argon ion laser, which delivered 2˜50 mW laser beam to the sample. Absorption spectra of silver particles were recorded by an Agilent UV-Visible spectrophotometer (Model 8453). The aggregate size of colloidal aggregates was determined by photon correlation spectroscopy (Malvern Zetasizer 3000HS). Electron microscopy images were recorded by a Hitachi scanning electron microscope (SEM).

Furthermore, the reagents and procedures adopted by the applicants in undertaking the investigation on the effect of cations and anions in chemical enhancement included screening multiple inorganic salts against target molecules: rhodamine 6G, deoxyribose nucleic acid (DNA) nucleotides, nucleosides, and bases. The tested salts were twelve alkali halides (lithium fluoride, lithium chloride, lithium bromide, lithium iodide, sodium fluoride, sodium chloride, sodium bromide, sodium iodide, potassium fluoride, potassium chloride, potassium bromide, and potassium iodide), in addition to hydrochloric acid and other salts such as beryllium chloride, magnesium chloride, calcium chloride, guanidine chloride, aluminum chloride, barium chloride, lanthanum chloride, and copper chloride. All reagents were of highest purity available (Sigma-Aldrich, St. Louis, Mo.).

Colloidal silver suspension was prepared by citrate reduction of silver nitrate as explained in Example 1.

For each measurement, the solution of target chemical species was first mixed with the silver colloids. A salt solution was then fully mixed into the silver colloid-target molecule mixture, and the spectrum was immediately collected. Mixing the salt solution with the silver colloids initiates the aggregation of the silver colloids, and the visible color change of the silver colloidal solution was observed by eye. The silver colloid aggregation was also observed by detecting a gradual shift of the absorption peak recorded by the UV-Visible spectrophotometer and by determining the hydrodynamic diameter of the aggregates using photon correlation spectroscopy.

At low concentrations, target molecules could adsorb to cuvette or tube walls, leading to erroneous measurements. In order to avoid this error, tubes were rinsed several times with equimolar sample solution to reach the adsorption equilibrium before measurements.

The results of the above-mentioned experiments could be summarized as follows. A strong SERS enhancement with LiCl was observed with dAMP. Spectra of dAMP in silver colloidal suspension, each mixed with LiCl and NaCl separately, are shown in FIG. 1C. The data show that the signal of dAMP increased by a factor of two by using LiCl instead of NaCl, which is a commonly used enhancer in many SERS studies. This led us to screen several salts for increased SERS enhancement effects.

The results from screening 12 salts (LiF, NaF, KF, LiCl, NaCl, KCl, LiBr, NaBr, KBr, LiI, NaI, and KI) for four chemical species: dAMP, deoxyadenosine, adenosine, and adenine were obtained. Raman peak at 734 cm⁻¹ due to the ring breathing mode was normalized so that its intensity with LiCl is 100. [dAMP]: 9 μM, [deoxyadenosine]: 9 μM, [adenosine]: 900 μM, and [adenine]: 900 nM. Final salt concentration was 180 mM. The target molecule concentration was adjusted to keep the signal of each molecule with several salts within the dynamic range of the detector. Introducing different cations affected the SERS signal significantly. LiCl consistently provided better SERS signal than NaCl. For example, the ratio of the SERS signal using LiCl versus NaCl for dAMP, deoxy-adenosine, adenosine and adenine were about 100/32, 100/28, 100/30 and 100/47, respectively. The strong enhancement of LiCl was also observed for various concentrations of target molecules. Lithium ion increased the SERS enhancements in the presence of chloride ions, while the same lithium ion reduces the SERS enhancements when mixed with fluoride ions.

FIG. 1D shows that the SERS spectrum of rhodamine 6G signal with LiCl is ten times stronger compared to that with NaCl. FIG. 1E also shows that the SERS spectrum of adenine is also stronger with LiCl than with NaCl. Thus, the strong enhancement effect with LiCl was observed for molecules that contain positive charge (rhodamine 6G), neutral charge (adenine, adenosine, deoxyadenosine), and negative charge (dAMP).

### Example 6

### New SERS Measurement Scheme to Enrich Analyte Molecules Between Metal Particle Junctions

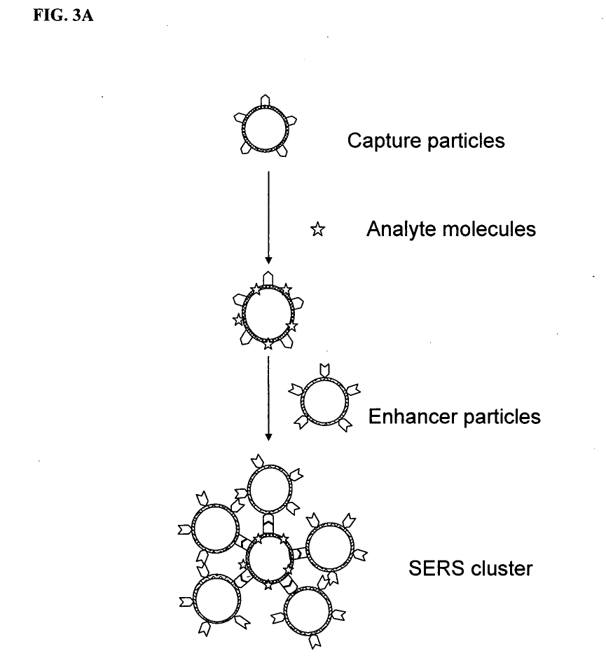

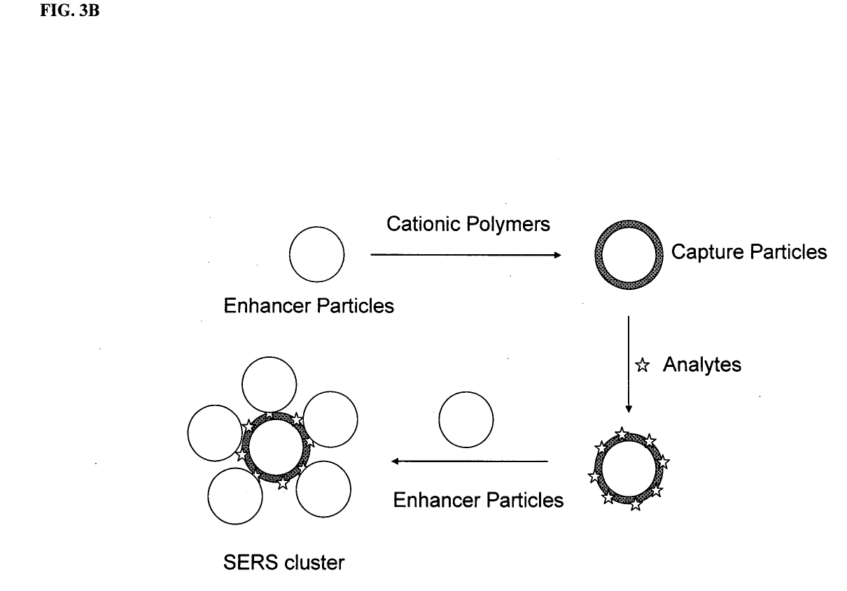

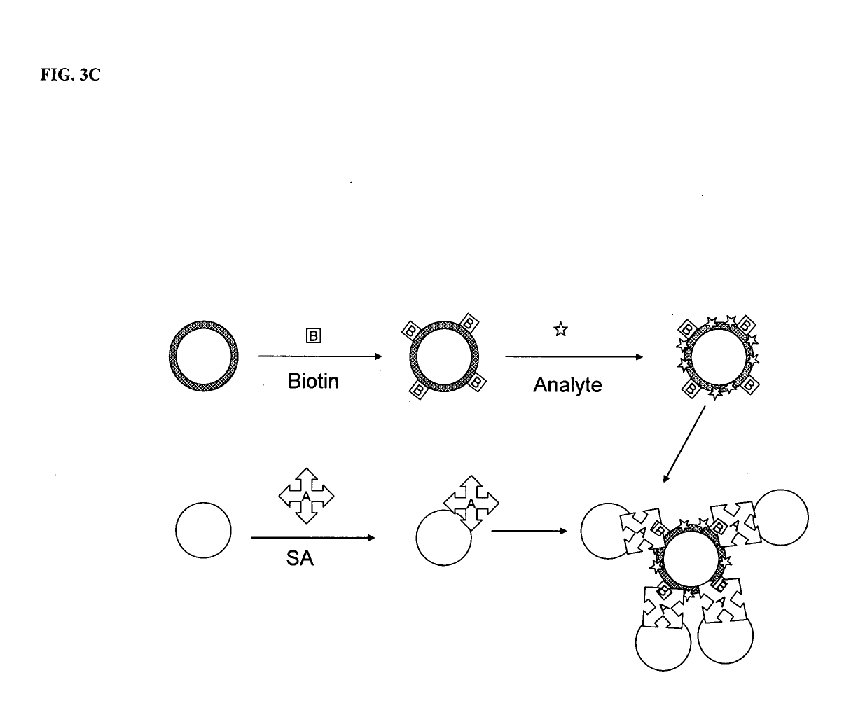

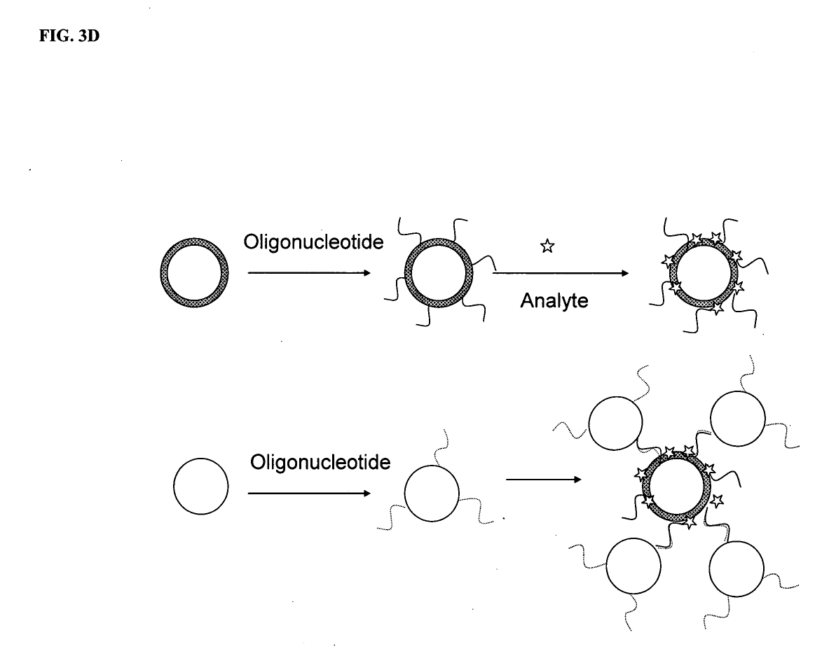

The embodiments of the invention propose new measurement platforms and schemes which could considerably improve the sensitivity by positioning the analyte molecules near the center of metal clusters where the enhancement effect is at the maximum. Moreover, by controlling the size and geometry of the clusters, Applicants expect to improve the reproducibility of the SERS measurements, making SERS a more reliable tool for quantitative analysis. The new SERS measurement scheme to enrich analyte molecules between metal particle junctions that could greatly improve the SERS signal intensity and consequently the detection limit for any types of analytes has been developed and explained below with reference to FIG. 3A.

To enrich analyte molecules between metal nanoparticles, the new SERS measurement scheme uses capture particles to adsorb the analyte molecules before enhancer particles are added to surround the enhancer particles. The capture particles are made of noble metal particles (such as Ag and Au) with desired surface properties to have high affinity for the analyte molecules. There are several ways to modify the nanoparticle surface to increase the adsorption of specific analyte molecules. A good example is to use cationic polymers to coat Au/Ag surface to create positively charged nanoparticles (capture particles) for negatively charged analytes.

Here is the typical experimental procedure with the new scheme:

(a) In a 0.5 mL centrifuge tube, add 50 μl of capture particles. The typical particle concentration is about 1.5×10¹¹ particles/mL for particles with an average diameter of 50 nm.
(b) Add 100 μL of samples to be analyzed. Mix the solution well with a pipette. Allow the capture particles and analyte molecules to reach adsorption equilibrium. If the driving force for adsorption is columbic interaction, the equilibrium can be reached in less than a minute. However, for adsorption based on specific molecular interaction such as antibody-antigen and complimentary DNA's, longer incubation time may be necessary.
(c) Add 50 μl of enhancer particles. Negatively metal particles (such as those stabilized by citrate) can be used as enhancer particles when the capture particles are positively charged. The concentration of the enhancer particles should be several times higher than that of capture particles to ensure most analyte molecules absorbed on the later could be situated at the junction of capture-enhancer particles.
(d) Transfer a small aliquot (100-200 μl) to sample holder (aluminum tray or 96-well) and collect Raman spectra.

Embodiments of FIGS. 3A to 3D

FIG. 3A describes a new measurement scheme which involves two steps: (1) mixing the analyte molecules with capture nanoparticles and (2) adding enhancer particles to cause cluster formation. The capture particle not only allows strong adsorption of analyte molecules on its surface, but also possesses functional groups and molecular moieties which can interact specifically with the enhancer particles. With the addition of a relatively large number of enhancer particles, the capture particle will be surrounded by the enhancer particles to form a SERS cluster. Thus, most analyte molecules could be either sandwiched between capture and enhancer particles or in the vicinity of the particle junctions.

The SERS cluster can be generated by using columbic interaction as shown in FIG. 3B. The capture particles are prepared by adsorption of cationic polymers (see Example 3). Co-polymers with desirable functional groups and/or hydrophobicity can be used so that the analyte molecules can be adsorbed strongly on the capture particles. Then negatively charged enhancer particles are added (wherein the citrate stabilized silver particles could be prepared using the method illustrated in Example 1). The electrostatic interaction between the capture and enhancer particles can cause rapid aggregation. In the presence of excessive amounts of enhancer particles, the SERS cluster will be spherical in shape with the capture particles at the center.

The strong Biotin-Streptavidin interaction is employed in forming SERS clusters in FIG. 3C. The capture particle is functionized with biotin while the enhancer particle is functionized with streptavidin (or vise versa). When the two types of particles are mixed, SERS clusters can be generated to provide strong signal enhancement for the analyte molecules on the capture particles. Similarly antibody-antigen interactions can be used to generate the SERS clusters.

Moreover, the interaction between complementary DNA/RNA strands can be utilized to cause SERS cluster formation as shown in FIG. 3D. The capture and enhancer particles can be derivatized with a segment of complimentary oligo-nucleic acids. For example, derivatization can be done by incubating the nanoparticles with a disulfide-protected oligonucleotide overnight. Sodium chloride is then gradually added over time to allow for maximal coverage of the nanoparticle surface with the highly-charged oligonucleotide. Great care must be taken to avoid aggregation of the nanoparticles by too large of an increase in the salt concentration. Unbound oligonucleotides can be removed by centrifugation and the pellet resuspended in a buffer with the desired sodium chloride concentration. After preparation of the capture and enhancer particles separately, the capture particles will be incubated with analyte molecules before enhancer particles are introduced to form SERS clusters.

### Example 7

### Target Specific Control and Generation of Hot Spots for Raman Tags

The embodiments of the invention utilize the advantage of ultra-sensitive SERS and incorporate biochemical specific particle aggregation towards biomolecule detection. This target specific event controls and generates hot spots for Raman tags during particle aggregation. The target specific control and generation of hot spots for Raman tags is achieved as follows with reference to FIGS. 4A to 4D.

The “hot spots” are junctions between nanoparticles. If the tag molecules are situated at the particle junctions, very strong SERS signals could result. Strong Raman signals can be obtained as the tag molecules have very high intrinsic Raman intensity. To ensure at least some tag molecules could be at the particle junctions, a large number of tag molecules could be attached to the nanoparticles.

By the embodiments of the invention, in particular, the embodiments of FIGS. 4A to 4D allow the determination of aggregation of tagged particles induced by analyte molecules (antibody, antigen, or DNA) by detecting a change in the SERS signal before and after aggregation. The tagged particle has both a Raman label and a probe molecule. The probe molecule hybridizes with the antibody or antigen. If aggregation happens, based on the knowledge of the probe, the antibody or antigen is determined.

Embodiment of FIG. 4A

FIG. 4A demonstrates how a target antibody is detected by SERS using this invention. A Raman tag and a specific antigen like a peptide are attached to the metal particle surface. The surface is then coated with hydrophilic layer using poly-ethylene glycol or bovine serum albumin (BSA) to prevent aggregation. The coating can be performed before the Raman tag and antigen attachment. Then a standard immunoassay procedure can be followed using these functional Raman particles. Aggregation occurs when target antibody is present, thus a strong SERS can be measured from the tag. In contrast, no aggregation occurs and no SERS is to be observed if the target antibody is absent.

FIG. 4A can also serve as an example for detecting HCV antibodies. HCV antigen is attached to silver particle. A Raman tag is also attached to the silver particle. The tag and the antigen could have a background Raman spectrum before the tagged particles aggregate. If there exit HCV antibodies, the antigen-antibody binding could cause silver particles to aggregate thus a strong (enhanced) Raman spectrum could appear to indicate the positive result.

Embodiment of FIG. 4B

FIG. 4B illustrates how multiple target antibodies in one sample can be detected at the same time by SERS using this invention. In this case, two or more types of tagged particles are used, each derivatized with a unique detection probe and a Raman label with a distinct spectrum for identification.

FIG. 4B is an example to detect multiple targets. Silver particle are individually labeled with HIV, HBV and HCV antigens. Different Raman tags are attached to the antigen labeled particle respectively. Existence of any specific antibody could cause a specific type of tagged particles to aggregate thus a specific Raman spectrum could appear to indicate the positive result.

Embodiment of FIG. 4C

FIG. 4C shows how one target antibody can be verified by duplication using the same detection probe but with different Raman tags. The mechanism of detection is similar to that in FIG. 4B except that two or more distinctive Raman tags are assigned to one detection probe.

FIG. 4C is an example to redundantly detect one specific target. Silver particles labeled with HCV antigen can be labeled with two or more different Raman tags. Two or more Raman spectra can be measured against one specific binding of HCV antigen and antibody to increase the credibility of the test.

Embodiment of FIG. 4D

FIG. 4D demonstrates how multiplexed DNA detection works. Raman particles are functionalized with different tags and different oligonucleotide probes. The individual Raman spectroscopic fingerprints (with narrow bands) of the particle probes can be identified by SERS after hybridization with the DNA targets.

FIG. 4D is an example to detect DNA hybridization. DNA hybridization is very base sequence specific. Several sets of probes can be designed to interrogate one target sequence. Perfect sequence match and mismatch can be designed to redundantly detect one specific target sequence. HIV, HBC and HCV target sequence are known in the data base. Probes can be designed according to their complementary sequence.

The embodiments of this invention have yet other several practical uses. For example, one embodiment of the invention allows molecules and nanomaterials detection/analysis based on the electrical readout of specific captured Raman signals (fingerprints) of molecules and nanomaterials. Another embodiment of the invention has potential applications for nanomaterials study to be used in electronic devices (transistors and interconnects) as well as well as for detection of bio-species (DNA, protein, viruses etc.) for molecular diagnostics, homeland security, drug discovery and life science R&D work.

This application discloses several numerical range limitations that support any range within the disclosed numerical ranges even though a precise range limitation is not stated verbatim in the specification because the embodiments of the invention could be practiced throughout the disclosed numerical ranges. Finally, the entire disclosure of the patents and publications referred in this application, if any, are hereby incorporated herein in entirety by reference.

1. A SERS cluster comprising a capture particle that is at least partially surrounded by analyte molecules, wherein both the capture particle and the analyte molecules surrounding the capture particle are at least partially surrounded by enhancer particles, wherein a majority of the analyte molecules are either sandwiched between capture and enhancer particles or located between junctions of the enhancer particles.

2. The SERS cluster of claim 1, wherein the capture particle comprises a compound that binds to enhancer particles by columbic interaction.

3. The SERS cluster of claim 1, wherein at least some of the analyte molecules comprise a compound that binds to capture particle by columbic interaction, covalent bonding or specific interaction.

4. The SERS cluster of claim 1, wherein the capture particle comprises a first molecule and the enhancer particle comprises a second molecule such that the first and second molecules have a specific interaction that causes the capture particle to bind to at least some of the enhancer particles.

5. The SERS cluster of claim 4, wherein the first molecule comprises Biotin and the second molecule comprises Streptavidin.

6. The SERS cluster of claim 4, wherein the first and second molecules comprise complimentary strands of DNA or RNA.

7. The SERS cluster of claim 1, wherein the enhancer particles comprise metal-containing particles.

8. The SERS cluster of claim 7, wherein the metal-containing particles are coated with an organic molecule, wherein the organic molecule comprises a moiety that has an affinity for the metal particles and another moiety that has an affinity for the analyte.

9. The SERS cluster of claim 8, wherein the metal-containing particles comprise a metal selected from the group consisting of silver, gold, platinum and combinations thereof.

10. The SERS cluster of claim 1, wherein substantially all of the analyte molecules are either sandwiched between capture and enhancer particles or located between junctions of the enhancer particles.

11. The SERS cluster of claim 1, wherein the capture particle comprises a linker or a probe molecule.

12. The SERS cluster of claim 1, wherein the cluster particle comprises a SERS active particle.

13. A method of manufacturing a SERS cluster, the method comprising mixing analyte molecules with a capture particle to form a mixture and subsequently adding enhancer particles to the mixture to cause formation of the SERS cluster comprising a majority of the analyte molecules either sandwiched between capture and enhancer particles or located between junctions of the enhancer particles.

14. The method of claim 13, wherein the SERS cluster is formed by a columbic interaction between the capture molecule and at least some of the analyte molecules.

15. The method of claim 13, wherein the capture particle comprises a first molecule and the enhancer particle comprises a second molecule such that the first and second molecules have a specific interaction causing the capture particle to bind to at least some of the enhancer particles.

16. The method of claim 15, wherein the first molecule comprises Biotin and the second molecule comprises Streptavidin.

17. The method of claim 15, wherein the first and second molecules comprise complimentary strands of DNA or RNA.

18. The method of claim 13, further comprising adding a deflocculating agent to the mixture prior to adding the enhancer particles to the mixture, wherein the deflocculating agent prevents aggregation of the capture particle with other capture particles in the mixture.

19. The method of claim 18, wherein the deflocculating agent comprises a biopolymer.

20. The method of claim 13, wherein both the capture particle and the analyte molecules surrounding the capture particle are at least partially surrounded by the enhancer particles.

21. A method of SERS measurement on a SERS cluster, the method comprising mixing analyte molecules with a capture particle to form a mixture, subsequently adding enhancer particles to the mixture to cause formation of the SERS cluster comprising a majority of the analyte molecules either sandwiched between capture and enhancer particles or located between junctions of the enhancer particles, and measuring a Raman signal emitted by the SERS cluster.

22. The method of claim 21, wherein the SERS cluster is formed by a columbic interaction between the capture particles and at least some of the enhancer particles.

23. The method of claim 21, wherein the capture particle comprises a first molecule and the enhancer particle comprises a second molecule such that the first and second molecules have a specific interaction causing the capture particle to bind to at least some of the enhancer particles.

24. The method of claim 23, wherein the first molecule comprises Biotin and the second molecule comprises Streptavidin.

25. The method of claim 23, wherein the first and second molecules comprise complimentary strands of DNA or RNA.

26. The method of claim 21, further comprising adding a deflocculating agent to the mixture prior to adding the enhancer particles to the mixture, wherein the deflocculating agent prevents aggregation of the capture particle with other capture particles in the mixture.

27. The method of claim 26, wherein the deflocculating agent comprises a biopolymer comprising a bovine serum albumin or poly-ethylene glycol.

28. The method of claim 21, wherein both the capture particle and the analyte molecules surrounding the capture particle are at least partially surrounded by the enhancer particles.

29. A SERS cluster comprising a tagged particle and an analyte.

30. The SERS cluster of claim 29, wherein the tagged particle comprises a linker having a specific biochemical binding capability.

31. A method for detection of a target molecule comprising attaching a tag molecule comprising a Raman active compound and a probe or a linker to a SERS active particle, coating the SERS active particle with a deflocculating agent, exposing a plurality of the SERS active particles to target molecules to cause aggregation of the plurality of the SERS active particles, and detecting a Raman signal emitted by the tag.

32. The method of claim 31, wherein the deflocculating agent forms a hydrophilic layer on a surface of the SERS active particle.

33. The method of claim 31, wherein the deflocculating agent comprises bovine serum albumin or poly-ethylene glycol.

34. The method of claim 31, wherein the target is an antibody or an antigen.

35. The method of claim 31, wherein multiple targets in one sample are detected at the same time.

36. The method of claim 31, wherein one target is verified by duplication using one probe or one linker and a plurality of tags.

37. The method of claim 31, wherein plurality of the SERS particle are functionalized with plurality of probes or linkers.

38. The method of claim 37, wherein the method allows multiplexed DNA or RNA detection.

39. The method of claim 19, wherein the biopolymer comprise a bovine serum albumin or poly-ethylene glycol.
